(19) United States
(12) Patent Application Publication
(10) Pub. No.: US 2026/0041871 A1
(43) Pub. Date: Feb. 12, 2026

(54) BREATHING AIR-DRIVEN MECHANICAL VENTILATOR

(71) Applicant: IPLEXXUS HOLDINGS, LLC, Freeland, MI (US)

(72) Inventor: David A. DZIOBA, Freeland, MI (US)

(21) Appl. No.: 19/292,223

(22) Filed: Aug. 6, 2025

Related U.S. Application Data
(60) Provisional application No. 63/679,775, filed on Aug. 6, 2024.
(60) Provisional application No. 63/811,011, filed on May 23, 2025.

(51) Int. Cl. A61M 16/10; A61M 16/04; A61M 16/06; A61M 16/20

(52) U.S. Cl. CPC A61M 16/10; A61M 16/0402; A61M 16/06; A61M 16/201

(57) ABSTRACT

A mechanical ventilator may include an oscillating flow controller and a respiratory interface fluidly connected to a bi-directional fluid port of the oscillating flow controller. The mechanical ventilator may be connected to a source of pressurized breathing air. Breathing air from the source of pressurized breathing air may drive the oscillating flow controller to periodically change state according to a predetermined cycle emulating a predetermined breathing pattern. The same breathing air may be provided to a user according to the predetermined cycle.

### CROSS-REFERENCE TO RELATED APPLICATIONS

This application claims benefit under 35 U.S.C. § 119 of: U.S. Provisional Patent Application No. 63/679,775, filed Aug. 6, 2024, and U.S. Provisional Patent Application No. 63/811,011, filed May 23, 2025, and incorporates by reference the disclosures thereof in their entireties. This application also incorporates by reference in its entirety the disclosure of U.S. patent application Ser. No. 19/081,559, filed Mar. 17, 2025.

### BACKGROUND OF THE DISCLOSURE

Mechanical ventilators are known in the art. A mechanical ventilator may be used to help a person breathe when the person is not able to do so on his or her own. Conventional mechanical ventilators typically are complex, expensive, heavy, and bulky. Given the expense in purchasing, maintaining, and storing mechanical ventilators, situations may arise where the need for them outstrips the supply thereof. This phenomenon became particularly evident during the early stages of the COVID-19 pandemic.

Also, conventional mechanical ventilators typically require electrical power to operate. As such, they typically are not sufficiently portable to be used outside a hospital setting. Thus, they typically are not available for use by emergency medical technicians (EMTs) or military medics in the field. Further, their complexity could hinder field use, even if electrical power were available to operate them there.

It would be desirable to provide a relatively low-cost mechanical ventilator that is portable, simple to use, and operable without electrical power.

### SUMMARY OF THE DISCLOSURE

The present disclosure is directed to a mechanical ventilator including an oscillating flow controller and a respiratory interface. In embodiments, the oscillating flow controller may include a supply port, a bi-directional port, and an exhaust port. The respiratory interface may include a breathing air conduit having a first end fluidly coupled to the bi-directional port and a second end configured to supply the breathing air from the bi-directional port to a respiratory system of a user. The oscillating flow controller may be configured to receive pressurized breathing air through the supply port, selectively output the pressurized breathing air through the bi-directional port, selectively receive the pressurized breathing air through the bi-directional port, and selectively output the pressurized breathing air though the exhaust port. The oscillating flow controller also may be configured to alternate between a first state in which the oscillating flow controller enables flow of the pressurized breathing air from the supply port through the bi-directional port to the breathing air conduit and disables flow of the pressurized breathing air from the supply port and the bi-directional port to the exhaust port, and a second state in which the oscillating flow controller enables flow of the pressurized breathing air from the breathing air conduit through the bi-directional port to the exhaust port and disables flow of the pressurized breathing air from the supply port to the bi-directional port and the exhaust port. The oscillating flow controller further may be configured to cyclically change state between the first state and the second state in response to the pressurized breathing air flowing therethrough.

In embodiments, the first respiratory interface may be a tracheal tube having a first end proximate the bi-directional port and a second end configured for intubation into the first user. The first tracheal tube may be configured to cyclically transfer the pressurized breathing air from the first end to the second end and to periodically transfer spent breathing air from the second end to the first end. A directional control valve may be fluidly connected between the bi-directional port and the first tracheal tube. The directional control valve may include a first port fluidly connected to the bi-directional port, a second port fluidly connected to the tracheal tube, and a third port fluidly connected to an environment external to the mechanical ventilator. The directional control valve may be operable between a first state in which the directional control valve is configured to enable fluid flow from the bi-directional port through the first port and the second port to the tracheal tube and to disable fluid flow through the third port, and a second state in which the directional control valve is configured to disable fluid flow through the first port and to enable fluid flow from the tracheal tube through the second port and the third port to the environment external to the mechanical ventilator. The directional control valve may be controlled by fluid pressure between the bi-directional port and the first port. The directional control valve may be in the first state when the fluid pressure between the bi-directional port and the first port is relatively high. The directional control valve may be in the second state when the fluid pressure between the bi-directional port and the first port is relatively low.

In embodiments, the mechanical ventilator also may include a second respiratory interface fluidly connected to the bi-directional port. The second respiratory interface may be configured to provide breathing air from the fluid supply port to a respiratory system of a second user.

In embodiments, the mechanical ventilator may include a second respiratory interface having a second breathing air conduit. The oscillating flow controller may include a second bi-directional port. The oscillating flow controller may be configured to selectively output the pressurized breathing air through the second bi-directional port and selectively receive the pressurized breathing air through the second bi-directional port. A first end of the second breathing air conduit may be fluidly coupled to the second bi-directional port and a second end of the second breathing air conduit may be configured to supply the breathing air from the second bi-directional port to a respiratory system of a second user. The oscillating flow controller may be configured to, in the first state, disable flow of the pressurized breathing air from the supply port through the second bi-directional port to the second breathing air conduit and enable flow of the pressurized breathing air from the second breathing air conduit through the second bi-directional port to the exhaust port. The oscillating flow controller also may be configured to, in the second state, enable flow of the pressurized breathing air through the second bi-directional port to the second breathing air conduit and disable flow of the pressurized breathing air from the second bi-directional port to the exhaust port.

In embodiments, the first respiratory interface may be a positive-pressure face mask.

In embodiments, the oscillating flow controller may be configured to change state between the first state and the second state according to a predetermined cycle corresponding to a predetermined breathing pattern.

The present disclosure also is directed to a method of mechanical ventilation which may include providing an oscillating flow controller which may include a supply port, a bi-directional port, and an exhaust port. The oscillating flow controller may be configured to receive breathing air through the supply port, selectively output the breathing air through the bi-directional port, selectively receive the breathing air through the bi-directional port, and selectively output the breathing air through the exhaust port. The oscillating flow controller also may be configured to alternate between a first state in which the oscillating flow controller enables flow of the breathing air from the supply port through the bi-directional port to the breathing air conduit and disables flow of the breathing air from the supply port and the bi-directional port to the exhaust port, and a second state in which the oscillating flow controller enables flow of the breathing air from the breathing air conduit through the bi-directional port to the exhaust port and disables flow of the breathing air from the supply port to the bi-directional port and the exhaust port. The oscillating flow controller further may be configured to cyclically change state between the first state and the second state in response to the breathing air flowing therethrough The method also may include providing a respiratory interface having a breathing air conduit, fluidly coupling a first end of the breathing air conduit fluidly to the bi-directional port, fluidly coupling a second end of the breathing air conduit to a respiratory system of a user, and fluidly coupling the supply port to a source of pressurized breathing air. In embodiments, the respiratory interface may be a tracheal tube. In embodiments, the respiratory interface may be a positive pressure mask.

In embodiments, the method also may include providing a second respiratory interface having a second breathing air conduit. The oscillating flow controller also may include a second bi-directional port. The oscillating flow controller may be configured to selectively output the pressurized breathing air through the second bi-directional port and selectively receive the pressurized breathing air through the second bi-directional port. A first end of the second breathing air conduit may be fluidly coupled to the second bi-directional port and a second end of the second breathing air conduit may be configured to supply the breathing air from the second bi-directional port to a respiratory system of a second user. The oscillating flow controller may be configured to: in the first state, disable flow of the pressurized breathing air from the supply port through the second bi-directional port to the second breathing air conduit and enable flow of the pressurized breathing air from the second breathing air conduit through the second bi-directional port to the exhaust port; and, in the second state enable flow of the pressurized breathing air through the second bi-directional port to the second breathing air conduit and disable flow of the pressurized breathing air from the second bi-directional port to the exhaust port. The oscillating flow controller may be configured to change state between the first state and the second state according to a predetermined cycle corresponding to a predetermined breathing pattern.

### BRIEF DESCRIPTION OF THE DRAWINGS

FIG. 1 is a side cross-sectional view of an illustrative oscillating flow controller having first and second armatures, first and second pilot chambers, and first and second movable pressure barriers, with the first and second armatures in respective first positions according to the present disclosure;

FIG. 2 is a side cross-sectional view of the oscillating flow controller of FIG. 1 with the first and second armatures in respective second positions;

FIG. 3A is a perspective view of the first and second armatures of the oscillating flow controller of FIG. 1;

FIG. 3B is a perspective view of the first and second armatures of the oscillating flow controller of FIG. 1 further showing first and second travel limiters operably associated with the second armature;

FIG. 3C is a perspective view of the first and second armatures and first and second travel limiters of the oscillating flow controller of FIG. 1 wherein the first and second movable pressure barriers are embodied as first and second flexible diaphragms connected to the first and second travel limiters, respectively;

FIG. 3D is a perspective view of the first and second movable pressure barriers and the first and second travel limiters of the oscillating flow controller of FIG. 1 apart from the first and second armatures thereof;

FIG. 4A is a side elevation view of the first and second armatures, the first and second movable pressure barriers, and the first and second travel limiters of the oscillating flow controller of FIG. 1, wherein the first and second movable pressure barriers are embodied as first and second flexible diaphragms;

FIG. 4B is a side elevation view of first and second armatures of the oscillating flow controller of FIG. 1, wherein the first and second movable pressure barriers are embodied as first and second flexible bellows;

FIG. 5A is a perspective view of the first armature of the oscillating flow controller of FIG. 1;

FIG. 5B is an exploded perspective view of the first armature of the oscillating flow controller of FIG. 1;

FIG. 6A is a perspective view of the second armature of the oscillating flow controller of FIG. 1;

FIG. 6B is an exploded perspective view of the second armature of the oscillating flow controller of FIG. 1;

FIG. 7 is a perspective view of a first permanent magnet of the first armature concentrically surrounding a second permanent magnet of the second armature of the oscillating flow controller of FIG. 1 wherein respective magnetic centers of the first and second permanent magnets are aligned;

FIG. 8A is a block diagram showing schematically the oscillating flow controller of FIG. 1 in combination with a source of pressurized fluid connected to a fluid supply port thereof, a first external accumulator connected to a first bi-directional fluid port thereof, and a second external accumulator connected to a second bi-directional fluid port thereof;

FIG. 8B is a block diagram similar to the block diagram of FIG. 8A, further showing a fluid outlet port of the oscillating flow controller fluidly coupled to a fluid inlet of the source of pressurized fluid and to a vacuum breaker;

FIG. 9 is a side cross-sectional view of the oscillating flow controller of FIG. 1 in combination with the source of pressurized fluid connected to the fluid supply port thereof, a first external accumulator connected to the first bi-directional fluid port thereof, and a second external accumulator connected to the second bi-directional fluid port thereof, with the first and second armatures of the oscillating flow controller in respect first positions;

FIG. 10 is a side cross-sectional view of the oscillating flow controller of FIG. 1 in combination with the source of pressurized fluid connected to the fluid supply port thereof, the first external accumulator connected to the first bi-directional fluid port thereof, and the second external accumulator connected to the second bi-directional fluid port thereof, with the first and second armatures of the oscillating flow controller in respect second positions;

FIGS. 11-18 are side cross-sectional views of the oscillating flow controller of FIG. 1 in an illustrative sequence of operational states when used in an illustrative manner in combination with the source of pressurized fluid and the first and second external accumulators, wherein:

FIG. 11 shows the oscillating flow controller of FIG. 1 in a first operational state;

FIG. 12 shows the oscillating flow controller of FIG. 1 in a second operational state;

FIG. 13 shows the oscillating flow controller of FIG. 1 in a third operational state;

FIG. 14 shows the oscillating flow controller of FIG. 1 in a fourth operational state;

FIG. 15 shows the oscillating flow controller of FIG. 1 in a fifth operational state;

FIG. 16 shows the oscillating flow controller of FIG. 1 in a sixth operational state;

FIG. 17 shows the oscillating flow controller of FIG. 1 in a seventh operational state;

FIG. 18 shows the oscillating flow controller of FIG. 1 in an eighth operational state;

FIGS. 19A-19D are timing diagrams reflecting illustrative pressurization and depressurization of one or more external accumulators fluidly connected to the oscillating flow controller of FIG. 1;

FIG. 20 shows schematically a first illustrative embodiment of a mechanical ventilator including the oscillating flow controller of FIG. 1 in combination with a first respiratory interface fluidly connected to the first bi-directional port of the oscillating flow controller, wherein the second bi-directional port of the flow control valve is blocked, and wherein a source of pressurized breathing air is fluidly connected to the fluid supply port of the oscillating flow controller;

FIG. 21 shows schematically the mechanical ventilator of FIG. 20 further including a pressure regulator fluidly connected between the source of pressurized breathing air and the fluid supply port of the oscillating flow controller;

FIG. 22 shows schematically a second illustrative embodiment of a mechanical ventilator including the oscillating flow controller of FIG. 1 in combination with a first respiratory interface fluidly connected to the first bi-directional port of the oscillating flow controller and a second respiratory interface fluidly connected to the second bi-directional port of the oscillating flow controller, wherein a source of pressurized breathing air is fluidly connected to the fluid supply port of the oscillating flow controller;

FIG. 23 shows schematically a third illustrative embodiment of a mechanical ventilator similar to the mechanical ventilator of FIG. 20 but further including a first directional control valve fluidly connected between the first bi-directional port of the oscillating flow controller and the first respiratory interface;

FIG. 24A shows schematically the directional control valve of FIG. 23 in a first operational state wherein the directional control valve is aligned to enable flow from the first bi-directional port of the oscillating flow controller to the first respiratory interface and to disable flow between the directional control valve and an exhaust region external to the directional control valve;

FIG. 24B shows schematically the directional control valve of FIG. 23 in a second operational state wherein the directional control valve is aligned to enable flow from the first respiratory interface to an exhaust region external to the directional control valve and to disable flow between the first bi-directional port of the oscillating flow controller and the directional control valve;

FIG. 25A is a timing diagram showing pressure vs time at the fluid supply port of the flow control valve;

FIG. 25B is a timing diagram showing pressure vs time at the first bi-directional fluid port of the flow control valve in response to pressure at the fluid supply port of the flow control valve as shown in FIG. 25A;

FIG. 25C is a timing diagram showing pressure vs time at the second bi-directional fluid port of the flow control valve in response to pressure at the fluid supply port of the flow control valve as shown in FIG. 25AB;

FIG. 26 shows schematically a fourth illustrative embodiment of a mechanical ventilator similar to the mechanical ventilator of FIG. 23 but further including a second respiratory interface and a corresponding second directional control valve fluidly connected to the first bi-directional port of the oscillating flow controller; and

FIG. 27 shows schematically a fifth illustrative embodiment of a mechanical ventilator similar to the mechanical ventilator of FIG. 26 but further including third and fourth respiratory interfaces and corresponding third and fourth directional control valves fluidly connected to the second bi-directional port of the oscillating flow controller.

### DETAILED DESCRIPTION OF THE DRAWINGS

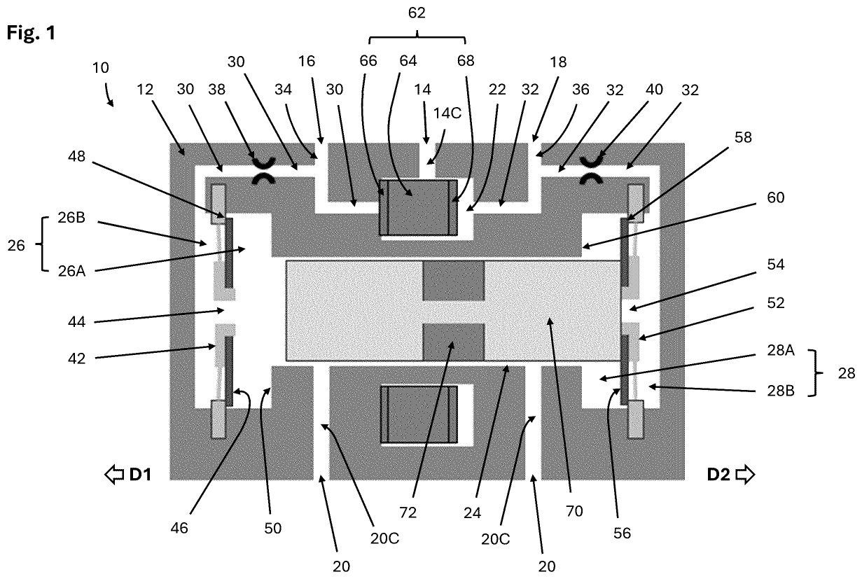

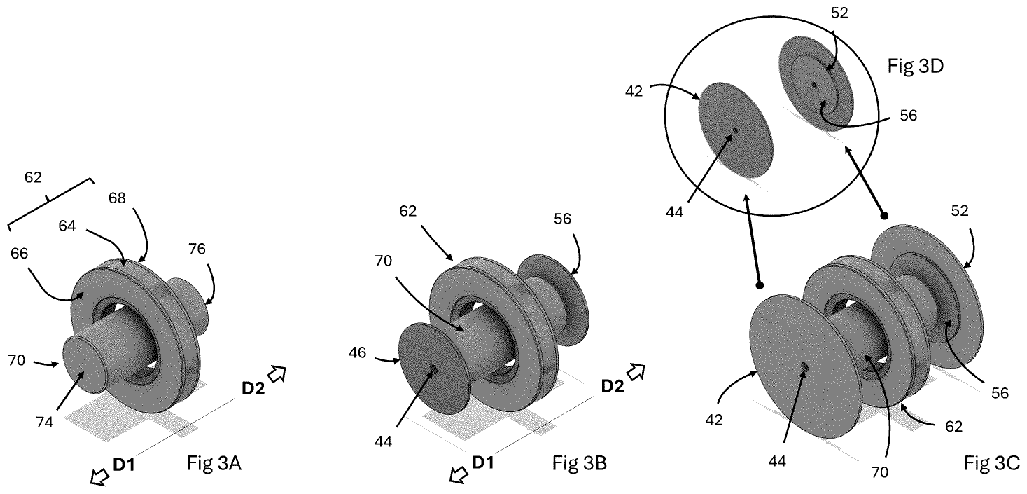

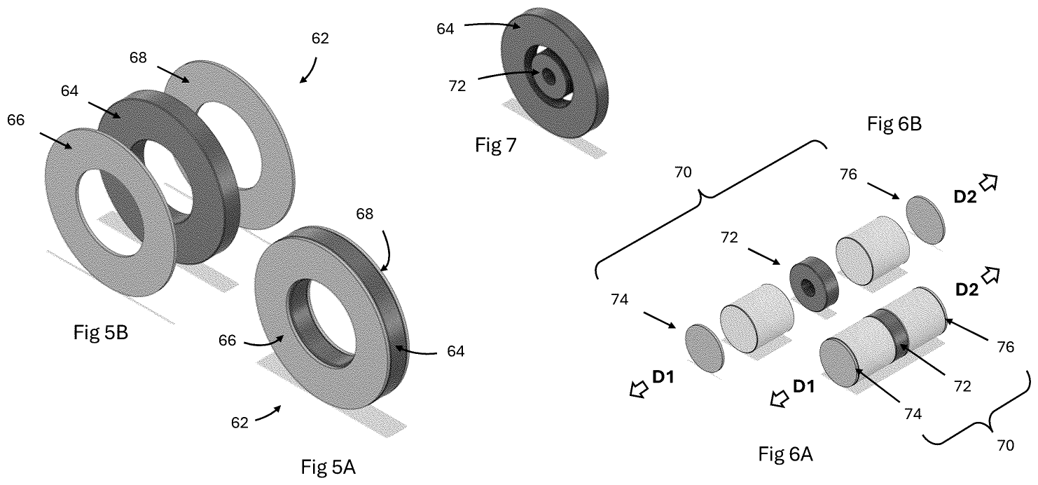

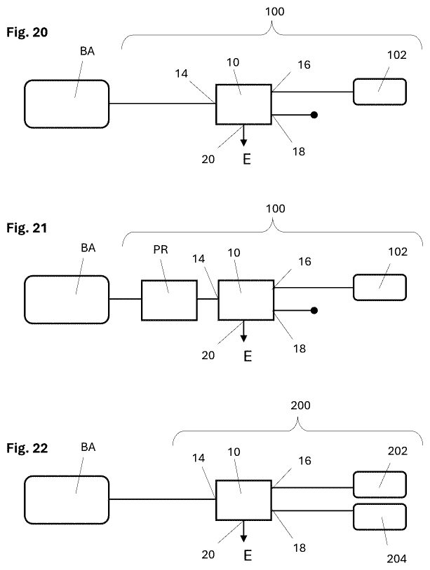

FIGS. 1-19D show an illustrative embodiment of an oscillating flow controller 10 (which may be referred to herein as an “oscillator”) and an illustrative use and operation of the same according to the present disclosure. FIGS. 20-27 show illustrative embodiments of mechanical ventilators including the oscillator 10 and one or more respiratory interfaces connected to one or more bi-directional ports thereof.

With reference initially to FIGS. 1-19D, the oscillator 10 includes a housing 12 defining a fluid supply port 14, a first bi-directional fluid port 16, a second bi-directional fluid port 18, a fluid exhaust port 20 (the drawings show two fluid exhaust ports 20 but only one is required), a first armature chamber 22 fluidly connected with the fluid supply port 14, a second armature chamber 24 fluidly connected with the fluid exhaust port(s) 20, a first cavity 26 fluidly connected with a first end of the second armature chamber 24, and a second cavity 28 fluidly connected with a second end of the second armature chamber 24. In embodiments, either or both of the first bi-directional port 16 and the second bi-directional port 18 may be eliminated, plugged, or otherwise deadheaded, as will be discussed further below.

As shown, the first armature chamber 22 is generally annular, and the second armature chamber 24 is stepped-cylindrical and generally concentric (or coaxial) with the first armature chamber 22. In embodiments, the first armature chamber 22 and the second armature chamber 24 may have other shapes. The first armature chamber 22 extends peripherally about at least a portion of the second armature chamber 24.

The first cavity 26 extends in a first axial direction D1 from a first end of the second armature chamber 24. The second cavity 28 extends in a second axial direction D2 from a second end of the second armature chamber 24. The first axial direction D1 is opposite the second axial direction D2.

A first fluid channel 30 fluidly connects the first armature chamber 22 with the first cavity 26. A second fluid channel 32 fluidly connects the first armature chamber 22 with the second cavity 28. A third fluid channel 34 fluidly connects the first fluid channel 30 with the first bi-directional port 16. In embodiments omitting the first bi-directional port 16, the third fluid channel 34 may be omitted, as well. A fourth fluid channel 36 fluidly connects the second fluid channel 32 with the second bi-directional port 18. In embodiments omitting the second bi-directional port 18, the fourth fluid channel 36 may be omitted, as well. A fluid exhaust channel 20C fluidly connects the second armature chamber 24 with the fluid exhaust port 20.

As shown, an optional first flow-restricting orifice 38 may be disposed in the first fluid channel 30, and an optional second flow-restricting orifice 40 may be disposed in the second fluid channel 32. The optional first Similarly, in embodiments, optional flow-restricting orifices could be installed in any fluid channel within the oscillator 10, for example, without limitation, any or all of the first and second fluid channels 30, 32, a fluid supply channel 14C and the fluid exhaust channel(s) 20C. In embodiments, any or all such flow-restricting orifices may be adjustable. Such optional flow-restricting orifices may be provided and sized as desired as factors contributing to the oscillation frequency of the flow oscillator 10, as will be discussed further below and as would be understood by one skilled in the art.

The oscillator 10 also includes a first movable pressure barrier 42 disposed in the first cavity 26 so as to divide the first cavity 26 into a first compartment 26A proximate the second armature chamber 24 and a second compartment 26B distant from the second armature chamber 24. The first movable pressure barrier 42 defines a first aperture 44 therein through which fluid may selectively flow between the first compartment 26A and the second compartment 26B, as will be discussed further below. At least a portion of the first movable pressure barrier 42 may be flexible and/or resilient.

Similarly, the oscillator 10 includes a second movable pressure barrier 52 disposed in the second cavity 28 so as to divide the second cavity 28 into a first compartment 28A proximate the second armature chamber 24 and a second compartment 28B distant from the second armature chamber 24. The second movable pressure barrier 52 defines a second aperture 54 therein through which fluid may selectively flow between the first compartment 28A and the second compartment 28B. At least a portion of the second movable pressure barrier 52 may be flexible and/or resilient.

The second compartment 26B of the first cavity 26 alone or in combination with the first fluid channel 30 and the third fluid channel 34 (if provided) comprises a first pilot chamber and may be referred to herein as the first pilot chamber 26B. Similarly, the second compartment 28B of the second cavity 28 alone or in combination with the second fluid channel 32 and the fourth fluid channel 36 (if provided) comprises a second pilot chamber and may be referred to herein as the second pilot chamber 28B.

The respective volumes of the first and second pilot chambers 26B, 28B may be sized as desired as factors contributing to the oscillation frequency of the oscillator 10, as will be discussed further below and as would be understood by one skilled in the art.

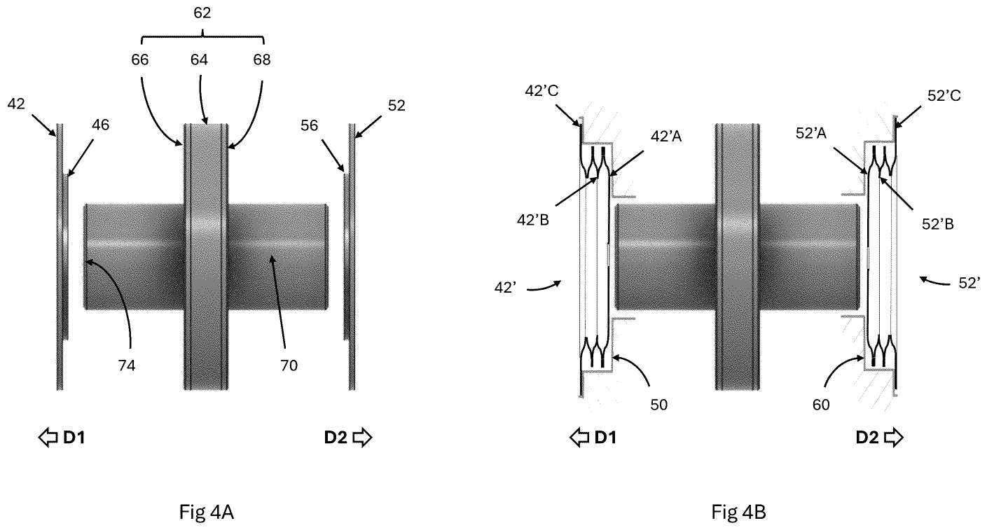

As shown generally in the drawings, the first and second movable pressure barriers 42, 52 may be embodied as first and second flexible diaphragms 42, 52. As shown in FIG. 4B, the first and second movable pressure barriers may be embodied as first and second flexible bellows 42′, 52′. In embodiments one of the first and second movable pressure barriers 42, 52 could be embodied as a flexible diaphragm, and the other could be embodied as a flexible bellows. One skilled in the art would recognize that the first and second movable pressure barriers 42, 52 could be embodied in other ways, as well.

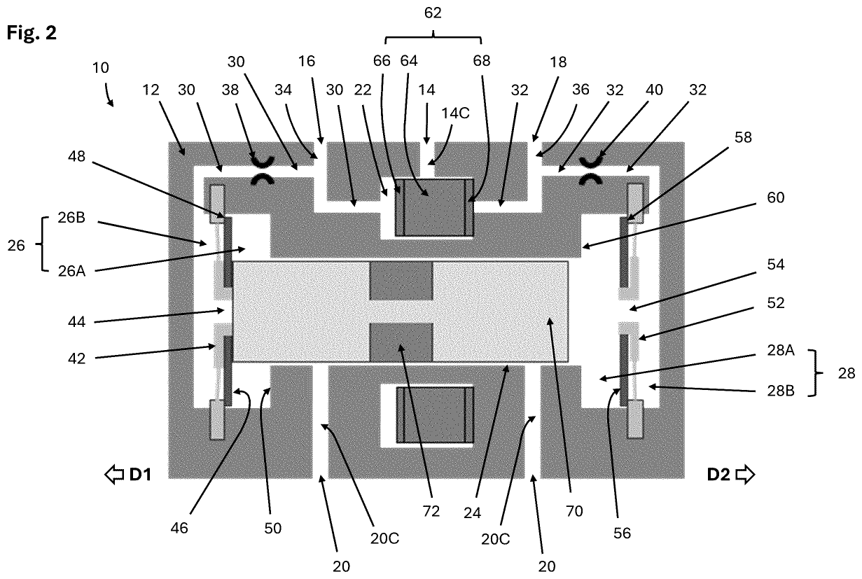

The oscillator 10 further includes a first armature 62 slidingly received within the first armature chamber 22. The first armature 62 has a first end and a second end opposite the first end. The first end of the first armature 62 faces the first direction D1, and the second end of the first armature 62 faces the second direction D2. A first permanent magnet 64 is disposed in a central region of the first armature 62 between the first and second ends thereof. The first permanent magnet 64 may be fixed to the first armature 62. The first end of the first armature 62 is configured to selectively and sealingly occlude the first fluid channel 30. For example, as may be best shown in FIG. 1, the first end of the first armature 62 is configured selectively and sealingly engage with a first wall of the first armature chamber 22 opposite the first end of the first armature and defining a corresponding end of the first fluid channel 30 to thereby occlude the first fluid channel 30. Similarly, the second end of the first armature 62 is configured to selectively and sealingly occlude the second fluid channel 32. For example, as may be best shown in FIG. 2, the second end of the first armature 62 is configured to selectively and sealingly engage with a second wall of the first armature chamber 22 opposite the second end of the first armature 62 and defining a corresponding end of the second fluid channel 32.

In embodiments, one or more seals may be provided to facilitate sealing engagement of the first armature 62 with the first and second walls of the first armature chamber 22. For example as shown, the first end of the first armature 62 includes a first face seal 66, and the second end of the first armature 62 includes a second face seal 68. In embodiments, the first face seal 66 may instead be integrated with the housing 12 opposite the first end of the first armature 62 and be configured to selectively and sealingly engage the first end of the first armature 62. Similarly, the second face seal 68 may instead be integrated with the housing 12 opposite the second end of the first armature 62 and be configured to selectively and sealingly engage the second end of the first armature 62. In embodiments, other forms of seals (not shown) may be provided in addition to or instead of the foregoing face seals.

The first armature 62 is configured to slide axially within the first armature chamber 22 in the first and second directions D1, D2 between a first position and a second position. The first armature 62 is configured to resist or block fluid flow between the first armature chamber 22 and the first fluid channel 30, and to enable flow between the first armature chamber 22 and the second fluid channel 32 when the first armature 62 is in the first position. Also, the first armature 62 is configured to enable fluid flow between the first armature chamber 22 and the first fluid channel 30, and to resist or block fluid flow between the first armature chamber 22 and the second fluid channel 32 when the first armature 62 is in the second position. More specifically, when the first armature 62 is in the first position, the first end of the first armature 62 (and the first face seal 66 if provided) engages a wall of the first armature chamber 22 defining the corresponding end of the first fluid channel 30 and occludes the end of the first fluid channel 30, while the second end of the first armature 62 is spaced from a wall of the first armature chamber 22 defining the corresponding end of the second fluid channel 32. When the first armature 62 is in the second position, the first end of the first armature 62 is spaced from the wall of the first armature chamber 22 defining the corresponding end of the first fluid channel 30, while the second end of the first armature 62 (including the second face seal 68 if provided) engages the wall of the first armature chamber 22 defining the corresponding end of the second fluid channel 32 and occludes the second fluid channel 32. As such, the first armature 62 and the housing 12 cooperate to define a first multi-port valve. Also, as is evident from the drawings, a radial clearance between the first armature 62 and the housing 12 is sufficient to enable substantial fluid flow through the first armature chamber 22, between the first armature 62 and the housing 12 defining the first armature chamber 22.

Similarly, the oscillator 10 includes a second armature 70 slidingly received within the second armature chamber 24. The second armature 70 has a first end and a second end opposite the first end. The first end of the second armature 70 faces the first direction D1, and the second end of the second armature 70 faces the second direction D2. A second permanent magnet 72 is disposed in a central region of the second armature 70 between the first and second ends thereof. The second permanent magnet 72 may be fixed to the second armature 70. The first end of the second armature 70 is configured to selectively engage with an adjacent face of the first movable pressure barrier 42 and to thereby selectively occlude the first aperture 44. Similarly, the second end of the second armature 70 is configured to selectively engage with an adjacent face of the second movable pressure barrier 52 and to thereby selectively occlude the second aperture 54. The first end of the second armature 70 may include a first face seal 74, and the second end of the second armature 70 may include a second face seal 76. In embodiments, the first face seal 74 may instead be integrated with the first movable pressure barrier 42 and configured to selectively and sealingly engage with the first end of the second armature 70. Similarly, the second face seal 76 may instead be integrated with the second movable pressure barrier 52 and configured to selectively and sealingly engage with the second end of the second armature 70. As such, the second armature 70, the first movable pressure barrier 42, and the second movable pressure barrier 52 cooperate to define a second multi-port valve. Also, as is evident from the drawings, a radial clearance between the second armature 70 and the housing 12 is sufficient to enable substantial fluid flow through the second armature chamber 24, between the second armature 70 and the housing 12 defining the second armature chamber 24.

The foregoing first and second multi-port valves and the housing may cooperate to define the first and second pilot chambers 26B, 28B. In embodiments including the first flow-restricting orifice 38, the first flow-restricting orifice 38 divides the first pilot chamber 26B into a first section proximate the first armature (and, therefore, the first multi-port valve) and a second section proximate the first movable pressure barrier 42 and the second armature 70 (and, therefore, the second multi-port valve). In embodiments including the second flow-restricting orifice 40, the second flow-restricting orifice 40 similarly divides the second pilot chamber 28B into a first section proximate the first armature (and, therefore, the first multi-port valve) and a second section proximate the first movable pressure barrier 42 and the second armature 70 (and, therefore, the second multi-port valve).

The second armature 70 is configured to slide axially within the second armature chamber 24 in the first and second directions D1, D2 between a first position and a second position. The second armature 70 is configured to resist or block fluid flow between the first and second compartments 28A, 28B of the second cavity 28 (and, therefore, between the second armature chamber 24 and the second pilot chamber 28B), and to enable flow between the first and second compartments 26A, 26B of the first cavity 26 (and, therefore, between the second armature chamber 24 and the first pilot chamber 26B) when the second armature 70 is in the first position. Also, the second armature 70 is configured to enable fluid flow between the first and second compartments 28A, 28B of the second cavity 28 (and, therefore, between the second armature chamber 24 and the second pilot chamber 28B), and to resist or block flow between the first and second compartments 26A, 26B of the first cavity 26 (and, therefore, between the second armature chamber 24 and the first pilot chamber 26B) when the second armature 70 is in the second position. More specifically, when the second armature 70 is in the first position, the second end of the second armature 70 (and the second face seal 76 if provided) engages the adjacent face of the second movable pressure barrier 52 and occludes the second aperture 54, while the first end of the second armature 70 is spaced from the first movable pressure barrier 42 and the first aperture 44. When the second armature 70 is in the second position, the second end of the second armature 70 is spaced from the second movable pressure barrier 52 and the second aperture 54, while the first end of the second armature 70 (including the first face seal 74 if provided) engages the adjacent face of the first movable pressure barrier 42 and occludes the first aperture 44.

In embodiments, the oscillator 10 may include a first travel limiter configured to restrict displacement of the first movable pressure barrier 42 relative to one or both of the first movable pressure barrier 42 and the housing 12. For example, and with reference to FIG. 4A, wherein the first movable pressure barrier 42 is shown as a first flexible diaphragm 42, the first travel limiter may be embodied as a first flange 46 adhered to or otherwise integrated with a central portion of the first flexible diaphragm 42 proximate the first aperture 44. The first flange 46 may be configured to engage a land 48 defined by a peripheral portion of the first flexible diaphragm 42 radially outward from the central portion and the first aperture 44 of the first flexible diaphragm 42 when the first flexible diaphragm 42 is displaced in the first direction D1, thereby limiting displacement of the first flexible diaphragm 42 in the first direction D1. Also, the housing 12 may define a land 50 configured to engage with the first flange 46 when the first flexible diaphragm 42 is displaced in the second direction D2, thereby limiting displacement of the first flexible diaphragm 42 in the second direction D2.

Similarly, the oscillator 10 may include a second travel limiter configured to restrict displacement of the second movable pressure barrier 52 relative to one or both of the second movable pressure barrier 52 and the housing 12. For example, and with continued reference to FIG. 4A, wherein the second movable pressure barrier 52 is shown as a second flexible diaphragm 52, the second travel limiter may be embodied as a second flange 56 adhered to or otherwise integrated with a central portion of the second movable pressure barrier 52 proximate the second aperture 54. The second flange 56 may be configured to engage a land 58 defined by a peripheral portion of the second movable pressure barrier 52 radially outward from the central portion and the second aperture 54 of the second movable pressure barrier 52 when the second movable pressure barrier 52 is displaced in the second direction D2, thereby limiting displacement of the second movable pressure barrier 52 in the second direction D2. Also, the housing 12 may define a land 60 configured to engage with the second flange 56 when the second movable pressure barrier 52 is displaced in the first direction D1, thereby limiting displacement of the second movable pressure barrier 52 in the first direction D1.

In embodiments wherein one or both of the first and second pressure barriers 42, 52 are first and second flexible bellows 42′, 52′, for example, as shown in FIG. 4B, respective portions of the respective flexible bellows 42′, 52′ may inherently function as first and second travel limiters in the directions of extension and compression of the respective flexible bellows 42′, 52′, as would be recognized by one skilled in the art. For example, respective innermost segments 42′A, 52′A of the first and second flexible bellows 42′, 52′ may be configured to contact the respective first and second lands 50, 60 when the first and second flexible bellows 42′, 52′ are extended and thus limit the extension thereof. Also, the respective innermost segments 42′A, 52′A of the first and second flexible bellows 42′, 52′ and intervening segments 42′B, 52′B of the first and second flexible bellows 42′, 52′ may be configured to stack up against respective outermost segments 42′C, 52′C of the first and second flexible bellows 42′, 52′ when the first and second flexible bellows 42′, 52′ are compressed. As shown in FIG. 4B, the direction of extension of the first flexible bellows 42′ is the second direction D2, and the direction of compression of the first flexible bellows 42′ is the first direction D1. As also shown in FIG. 4B, the direction of extension of the second flexible bellows 52′ is the first direction D1, and the direction of compression of the second flexible bellows 52′ is the second direction D2.

As mentioned above, the first armature 62 includes a first magnet 64, and the second armature 70 includes a second magnet 72. As shown, the first magnet 64 surrounds and is generally coaxial with the second magnet 72. Also, as best shown in, for example, FIGS. 1 and 2, respectively, the first magnet 64 is axially offset from the second magnet 72 in the first direction D1 when the first armature 62 and the second armature 70 are in their respective first positions, and the first magnet 64 is axially offset from the second magnet 72 in the second direction D2 when the first armature 62 and the second armature 70 are in their respective second positions. More specifically, a magnetic center of the first magnet 64 is axially offset from a magnetic center of the second magnet 70 in the first direction D1 when the first and second armatures 62, 70 are in their respective first positions, and the magnetic center of the first magnet 64 is axially offset from the magnetic center of the second magnet 70 in the second direction D2 when the first and second armatures 62, 70 are in their respective second positions.

As suggested above, the first and second magnets 62, 70 cooperate to define a biasing mechanism configured to simultaneously bias both the first and second armatures 62, 70 (and, therefore the first and second multi-port valves they respective define) toward their respective first positions or their respective second positions. More specifically, the first and second magnets 64, 72 are configured so that a magnetic field between the first and second magnets 64, 72 causes the first and second magnets 64, 72 to repel each other at least axially. The strength of the magnetic field and, therefore, the repulsive force, is greatest when the magnetic centers of the first and second magnets 62, 70 are nearest to each other. Conversely, the strength of the magnetic field and, therefore, the repulsive force, is lowest when the magnetic centers of the first and second magnets 62, 70 are farthest from each other. As such, the magnetic field biases the first and second armatures 62, 70 toward their respective first positions when the first magnet 64 is axially offset from the second magnet 72 in the first direction D1. Similarly, the magnetic field biases the first and second armatures 62, 70 toward their respective second positions when the first magnet 64 is axially offset from the second magnet 72 in the second direction D2. Thus, in the absence of other forces acting on the first and second armatures 62, 70, the first and second armatures 62, 70 are stable when both of the first and second armatures 62, 70 are in either their respective first positions or their respective second positions. The respective field strengths of the first and second magnets 64, 72 may be selected as desired as factors contributing to the oscillation frequency of the flow oscillator 10, as would be recognized by one skilled in the art. As shown, the first and second magnets 64, 72 are located at generally central portions of the first and second armatures 62, 70, respectively. In embodiments, the first and second magnets 64, 72 could be located elsewhere with respect to the first and second armatures 62, 70, respectively, with the magnetic centers of the first and second magnets 64, 72 configured to interact with each other as described above. For example, the first and second magnets 64, 72 could be located proximate the respective first ends or second ends of the first and second armatures 62, 70. In embodiments, the foregoing magnetic biasing mechanism could be replaced with another magnetic biasing mechanism or a non-magnetic biasing mechanism configured to simultaneously bias the first and second armatures 62, 70 towards their respective first positions or their respective second positions.

As mentioned above, the volumes of the first and second pilot chambers as described above, the selection of optional orifices in and or all of the first, second, third, and fourth fluid channels 30, 32, 34, 36 and the exhaust channel(s) 20C, and the magnetic field strength between the first and second magnets 64, 72 are factors that contribute to the oscillation frequency of the oscillator 10. Other factors may contribute to the oscillation frequency of the oscillator 10, including without limitation: the respective sizes of the fluid supply port 14, the first and second bi-directional ports 16, 18, and the fluid exhaust port(s) 20; the respective sizes of the first and second apertures 44, 54; the respective clearances between the second armature 70 and the first and second apertures 44, 54; the clearance between the second armature 70 and the portion of the housing 12 defining the second armature chamber 24; the respective clearances between the first armature 62 and the portions of the housing 12 defining the corresponding adjacent ends of the first and second flow channels 30, 32; and the movable surface area of the first and second movable pressure barriers 42, 52. One skilled in the art would understand how to select at least the foregoing components or features in order to achieve a desired oscillation frequency of the oscillator 10, as will be discussed further below.

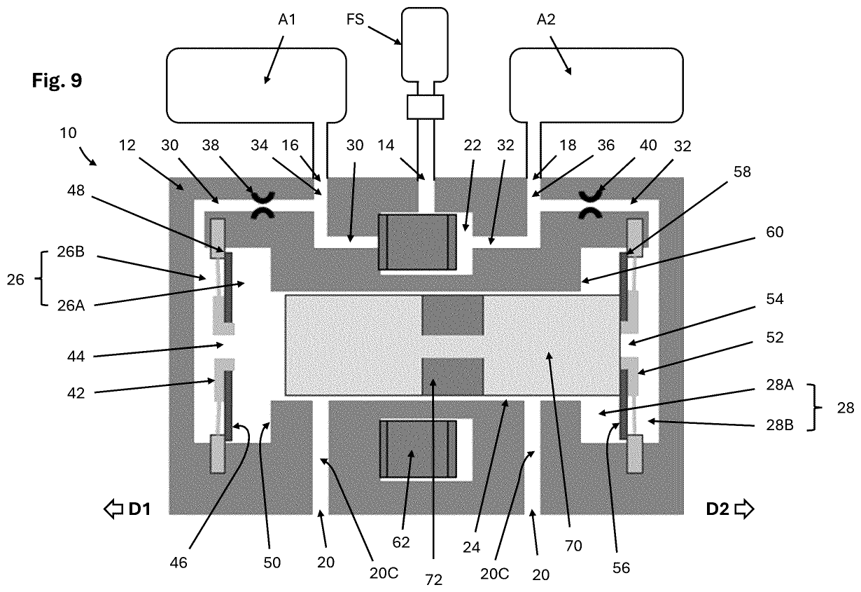

As best shown in FIGS. 9 and 10, the oscillator 10 is configured for use in connection with a source of pressurized fluid FS in fluid communication with the fluid supply port 14, a first external accumulator A1 in fluid communication with the first bi-directional fluid port 16, and a second external accumulator A2 in fluid communication with the second bi-directional fluid port 18. The pressurized fluid may be a liquid or a gas, for example, air. The first and second external accumulators A1, A2 may be, without limitation, first and second selectively inflatable compartments of a therapeutic support surface overlay (not shown). One skilled in the art would recognize that the respective volumes and internal flow characteristics of the first and second external accumulators A1, A2, as well the respective volumes and flow characteristics of fluid conduits connecting the first and second external accumulators A1, A2, respectively, to the first and second bi-directional ports 16, 18 are factors that may contribute to the oscillation frequency of the flow oscillator 10.

FIG. 9 shows the oscillator 10 in a first, stable state, with no fluid flowing from the source of pressurized fluid FS and with the first and second external accumulators A1, A2 at an ambient pressure. In this first, stable state, the first and second armatures 62, 70 are in their respective first positions and are biased to their respective first positions by the magnetic biasing force between the first and second magnets 64, 72. As such, the first armature 62 enables fluid flow from the first armature chamber 22 into the second fluid channel 32 and, therefore, to the second pilot chamber 28B, the fourth fluid channel 36, and the second bi-directional fluid port 18, while blocking fluid flow from the first armature chamber 22 into the first fluid channel 30 and, therefore, to the first pilot chamber 26B, the third fluid channel 34, and the first bi-directional fluid port 16. Also, the second armature 70 enables flow from the first fluid channel 30 through the first aperture 44 of the first movable pressure barrier 42, while resisting or blocking flow from the second fluid channel 32 through the second aperture 54 of the second movable pressure barrier 52. Thus, in this first, stable state, the interior portion of the housing 12 including the second armature chamber 24, the first pilot chamber 26B, and the first and third fluid channels 30, 34 are vented via the exhaust port 20 to an environment, for example, the atmosphere, surrounding the oscillator 10 and thus are at an ambient pressure.

FIG. 10 shows the oscillator 10 in a second, stable state, with no fluid flowing from the source of pressurized fluid FS and with the first and second external accumulators A1, A2 at an ambient pressure. In this second, stable state, the first and second armatures 62, 70 are in their respective second positions and are biased to their respective second positions by the magnetic biasing force between the first and second magnets 64, 72. As such, the first armature 62 blocks fluid flow from the first armature chamber 22 into the second fluid channel 32 and, therefore, to the second pilot chamber 28B, the fourth fluid channel 36, and the second bi-directional fluid port 18, while enabling fluid flow from the first armature chamber 22 into the first fluid channel 30 and, therefore, to the first pilot chamber 26B, the third fluid channel 34, and the first bi-directional fluid port 16. Also, the second armature 70 blocks flow from the first fluid channel 30 through the first aperture 44 of the first movable pressure barrier 42, while enabling flow from the second fluid channel 32 through the second aperture 54 of the second movable pressure barrier 52. Thus, in this second, stable state, the interior portion of the housing 12 including the second armature chamber 24, the second pilot chamber 28B, and the second and fourth fluid channels 32, 36 are vented via the exhaust port 20 to an environment, for example, the atmosphere, surrounding the oscillator 10 and thus is at an ambient pressure.

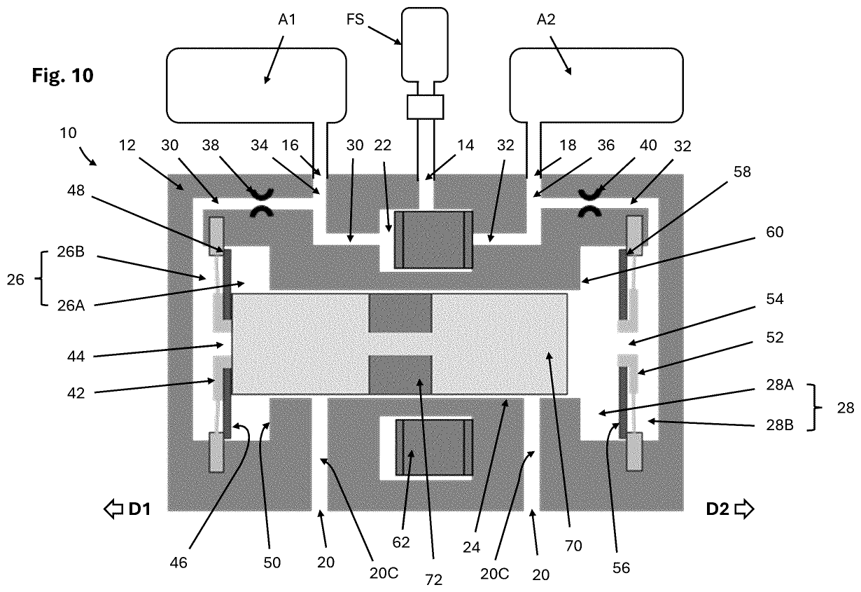

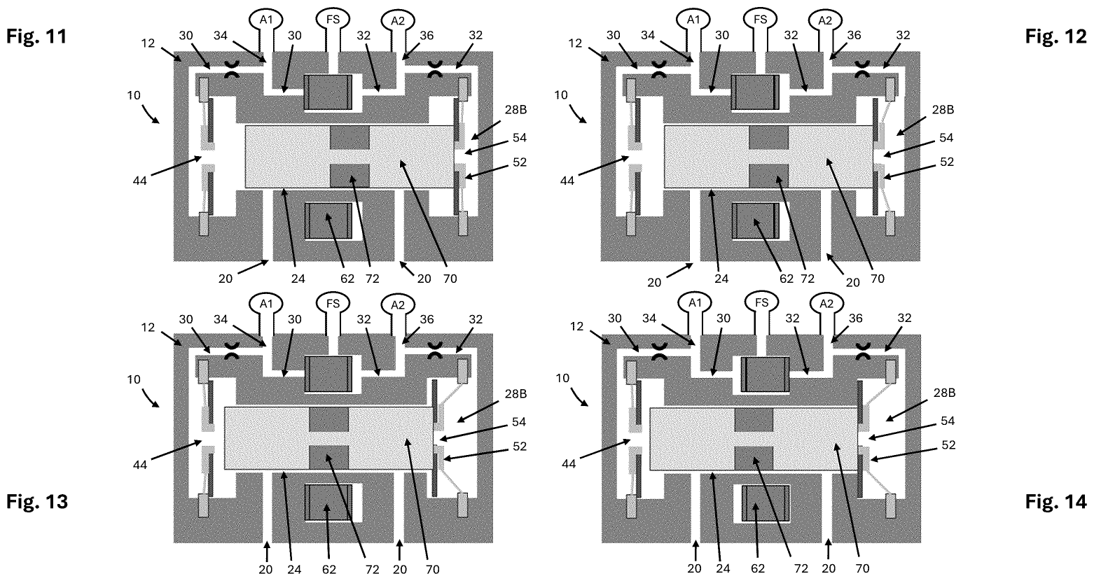

FIGS. 11-18 show the oscillator 10 connected to and receiving pressurized fluid from the source of pressurized fluid FS and selectively directing the pressurized fluid to or relieving the pressurized fluid from the first and second external accumulators A1, A2 in various operational states.

FIG. 11 shows the oscillator 10 in a first initial pressurization state, wherein the first and second armatures 62, 70 are in their respective first positions, as discussed further above and shown in FIG. 9. In this state, the oscillator 10 receives pressurized fluid from the source of pressurized fluid FS via the fluid supply port 14 and directs the pressurized fluid to: (i) the second pilot chamber 28B via the first armature chamber 22 and the second fluid channel 32; and (ii) the second external accumulator A2 via the first armature chamber 22, the second and fourth fluid channels 32, 36 and the second bi-directional fluid port 18. The foregoing flow of pressurized fluid causes the fluid pressures within the second external accumulator A2 and the second pilot chamber 28B to increase. One skilled in the art would recognize that the second and fourth channels 32, 36, and the second flow-restricting orifice 40 (if provided) may be configured so that the pressurized fluid enters the second pilot chamber 28B at a similar rate or at a substantially different rate (for example, more slowly) than that at which it enters the second external accumulator A2 and, therefore, that the fluid pressure within the second pilot chamber 28B increases at a similar rate or a substantially different rate than does the fluid pressure within the second external accumulator A2. Also, in the first initial pressurization state of FIG. 11, the first armature 62 disables flow from the first armature chamber 22 to and downstream of the first fluid channel 30 and, therefore, disables flow of pressurized fluid from the source of pressurized fluid FS to the first accumulator A1 and the first pilot chamber 26B. Further, the second armature 70 blocks fluid flow through the second aperture 54 and enables fluid flow through the first aperture 44, thereby enabling flow of pressurized fluid from the first external accumulator A1 through the fluid exhaust port(s) 20 via the third fluid channel 34, the first fluid channel 30, the first pilot chamber 26B, the second armature chamber 24 and the fluid exhaust channel(s) 20C.

FIG. 12 shows the oscillator 10 in a first intermediate pressurization state similar to the first initial pressurization state of FIG. 11 wherein the first armature 62 remains in its first position, but wherein increasing fluid pressure within the second chamber 28B has caused the second movable pressure barrier 52 to move in the first direction D1 and thereby displace the second armature 70 in the first direction D1. Consequently, the magnetic center of the second magnet 72 has been displaced in the first direction D1 toward, but not to, the magnetic center of the first magnet 64. Therefore, the repulsive magnetic force between the first magnet 64 and the second magnet 72 continues to bias the first magnet 64 (and thus the first armature 62) in the first direction D1, and to bias the second magnet 72 (and thus the second armature 70) in the second direction D2. (Because the first and second magnets 64, 72 are closer together in the first intermediate pressurization state of FIG. 12 compared to the first initial pressurization state of FIG. 11, the repulsive magnetic biasing force between the first and second magnets 64, 72, is greater in the first intermediate pressurization state FIG. 12 state than in the first initial pressurization state of FIG. 11, thus increasing the sealing pressure at the interface between the second end of the second armature 70 and the second movable pressure barrier 52.) As such, the alignment of fluid paths within the oscillator 10 remains as in the first initial pressurization state of FIG. 11. It follows that pressurized fluid continues to flow into the second external accumulator A2 and into the second pilot chamber 28B, the first armature 62 continues to disable flow of pressurized fluid from the source of pressurized fluid FS to and downstream of the first fluid channel 30, and the second armature 70 continues to enable fluid flow from the first external accumulator A1 to the environment surrounding the oscillator 10.

FIG. 13 shows the oscillator 10 in a second intermediate pressurization state similar to the first intermediate pressurization state of FIG. 12 wherein the first armature 62 remains in its first position, but wherein increasing fluid pressure within the second pilot chamber 28B has caused the second movable pressure barrier 52 to move further in the first direction D1 and thereby displace the second armature 70 further in the first direction D1 than in the first intermediate pressurization state of FIG. 12. Consequently, the magnetic center of the second magnet 72 has been displaced in the first direction D1 substantially to, but not beyond, the magnetic center of the first magnet 64. (Because the first and second magnets 64, 72 are closer together in the second intermediate pressurization state of FIG. 12 compared to the first intermediate pressurization state of FIG. 12, the magnetic biasing force between the first and second magnets 64, 72, is even greater in the second intermediate pressurization state FIG. 12 state than in the first intermediate pressurization state of FIG. 12, thus further increasing the sealing pressure at the interface between the second end of the second armature 70 and the second movable pressure barrier 52.) In this state, the magnetic biasing force between the first magnet 64 and the second magnet 72 destabilizes the first magnet 64 with respect to the second magnet 72. Notwithstanding, in the second intermediate pressurization state of FIG. 13, the alignment of fluid paths with the oscillator 10 remains as in the first initial pressurization state of FIG. 11. As such, pressurized fluid continues to flow into the second external accumulator A2 and into the second pilot chamber 28B, the first armature 62 continues to disable flow of pressurized fluid from the source of pressurized fluid FS to and downstream of the first fluid channel 30, and the second armature 70 continues to enable fluid flow from the first external accumulator A1 to the environment surrounding the oscillator 10.

FIG. 14 shows the oscillator 10 in a first transition state wherein the oscillator 10 transitions between the states shown in FIGS. 9 and 10, respectively. More specifically, FIG. 14 shows the oscillator 10 in a state wherein continued increasing fluid pressure within the second pilot chamber 28B has caused the second movable pressure barrier 52 to move still further in the first direction D1 (to the greatest extent permitted by the interaction of the second 56 with the land 60) and to thereby displace the second armature 70 still further in the first direction D1 than in the second intermediate pressurization state of FIG. 13. As shown in FIG. 14, the magnetic center of the second magnet 72 has been displaced in the first direction D1 beyond the magnetic center of the first magnet 64. As such, the direction of the magnetic biasing force between the first magnet 64 and the second magnet 72 has been reversed so that the magnetic biasing force biases the first magnet 64 in the second direction D2, and so that it biases the second magnet 72 in the first direction D1. Indeed, FIG. 14 shows the first armature 62 having been biased to its second position as shown in FIG. 10, and it shows the second armature 70 being biased toward, but not having been biased to, its second position.

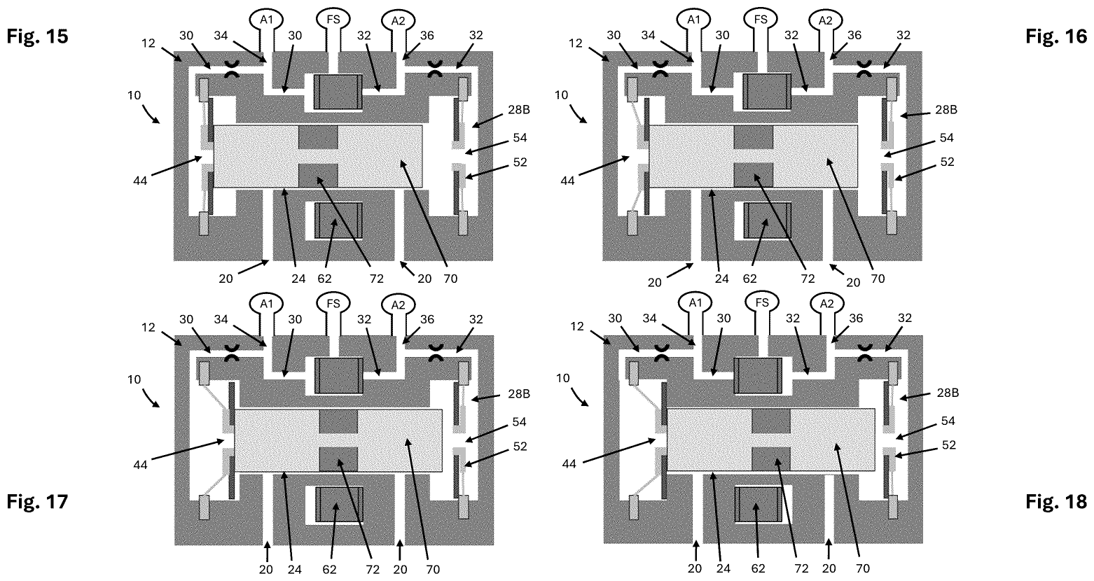

FIG. 15 shows the oscillator 10 in a second initial pressurization state, wherein the first and second armatures 62, 70 are in their respective second positions, as discussed further above and shown in FIG. 10. In this state, the oscillator 10 receives pressurized fluid from the source of pressurized fluid FS via the fluid supply port 14 and directs the pressurized fluid to: (i) the first pilot chamber 26B via the first armature chamber 22 and the first fluid channel 30; and (ii) the first external accumulator A1 via the first armature chamber 22, the first fluid channel 30, the third fluid channel 34, and the first bi-directional fluid port 16. The foregoing flow of pressurized fluid causes the fluid pressures within the first external accumulator A1 and the first pilot chamber 26B to increase. One skilled in the art would recognize that the first and third fluid channels 30, 34, and the first flow-restricting orifice 38 (if provided) may be configured so that the pressurized fluid enters the first pilot chamber 26B at a similar rate or at a substantially different rate (for example, more slowly) than it enters the first external accumulator A1 and, therefore, that the fluid pressure within the first pilot chamber 26B increases at a similar rate or a substantially different rate than does the fluid pressure within the first external accumulator A1. Also, in the second initial pressurization state of FIG. 15, the first armature 62 disables flow from the first armature chamber 22 to the second fluid channel 32 and, therefore, disables flow of pressurized fluid from the source of pressurized fluid FS to the second external accumulator A2 and the second pilot chamber 28B. Further, the second armature 70 blocks fluid flow through the first aperture 44 and enables fluid flow through the second aperture 54, thereby enabling flow of pressurized fluid from the second external accumulator A2 through the fluid exhaust port(s) 20 via the fourth fluid channel 36, the second fluid channel 32, the second pilot chamber 28, the second armature chamber 24 and the fluid exhaust channel(s) 20C.

FIG. 16 shows the oscillator 10 in a third intermediate pressurization state similar to the second initial pressurization state of FIG. 11 wherein the first armature 62 remains in its second position, but wherein increasing fluid pressure within the first pilot chamber 26B has caused the first movable pressure barrier 42 to move in the second direction D2 and thereby displace the second armature 70 in the second direction D2. Consequently, the magnetic center of the second magnet 72 has been displaced in the second direction D2 toward, but not to, the magnetic center of the first magnet 64. Therefore, the magnetic force between the first magnet 64 and the second magnet 72 continues to bias the first magnet 64 (and thus the first armature 62) in the second direction D2, and to bias the second magnet 72 (and thus the second armature 70) in the first direction D1. (Because the first and second magnets 64, 72 are closer together in the third intermediate pressurization state of FIG. 16 compared to the second initial pressurization state of FIG. 15, the magnetic biasing force between the first and second magnets 64, 72, is greater in the third intermediate pressurization state FIG. 16 state than in the second initial pressurization state of FIG. 15, thus increasing the sealing pressure at the interface between the second end of the second armature 70 and the second movable pressure barrier 52.) As such, the alignment of fluid paths within the oscillator 10 remains as in the second initial pressurization state of FIG. 15. It follows that pressurized fluid continues to flow into the first external accumulator A1 and into the first pilot chamber 26B, the first armature 62 continues to disable flow of pressurized fluid from the source of pressurized fluid FS to and downstream of the second fluid channel 32, and the second armature 70 continues to enable fluid flow from the second external accumulator A2 to the environment surrounding the oscillator 10.

FIG. 17 shows the oscillator 10 in a fourth intermediate pressurization state similar to the third intermediate pressurization state of FIG. 16 wherein the first armature 62 remains in its second position, but wherein increasing fluid pressure within the first pilot chamber 26B has caused the first movable pressure barrier 42 to move further in the second direction D2 and thereby displace the second armature 70 further in the second direction D2 than in the third intermediate pressurization state of FIG. 16. Consequently, the magnetic center of the second magnet 72 has been displaced in the second direction D2 substantially to, but not beyond, the magnetic center of the first magnet 64. (Because the first and second magnets 64, 72 are closer together in the fourth intermediate pressurization state of FIG. 17 compared to the third intermediate pressurization state of FIG. 16, the magnetic biasing force between the first and second magnets 64, 72, is even greater in the fourth intermediate pressurization state FIG. 17 than in the third intermediate pressurization state of FIG. 16, thus further increasing the sealing pressure at the interface between the second end of the second armature 70 and the second movable pressure barrier 52.) In this state, the magnetic biasing force between the first magnet 64 and the second magnet 72 destabilizes the first magnet 64 with respect to the second magnet 72. Notwithstanding, in the fourth intermediate pressurization state of FIG. 17, the alignment of fluid paths with the oscillator 10 remains as in the second initial pressurization state of FIG. 15. As such, pressurized fluid continues to flow into the first external accumulator A1 and into the first pilot chamber 26B, the armature 62 continues to disable flow of pressurized fluid from the source of pressurized fluid FS to and downstream of the second fluid channel 32, and the second armature 70 continues to enable fluid flow from the second external accumulator A2 to the environment surrounding the oscillator 10.

FIG. 18 shows the oscillator 10 in a second transition state wherein the oscillator 10 transitions between the states shown in FIGS. 10 and 9, respectively. More specifically, FIG. 18 shows the oscillator 10 in a state wherein continued increasing fluid pressure within the first pilot chamber 26B has caused the first movable pressure barrier 42 to move still further in the second direction D2 (to the greatest extent permitted by the interaction of the first travel limiter 46 with the land 50) and to thereby displace the second armature 70 still further in the second direction D2 than in the fourth intermediate pressurization state of FIG. 17. As shown in FIG. 18, the magnetic center of the second magnet 72 has been displaced in the second direction D2 beyond the magnetic center of the first magnet 64. As such, the direction of the magnetic biasing force between the first magnet 64 and the second magnet 72 has been reversed so that the magnetic biasing force biases the first magnet 64 in the first direction D1, and so that it biases the second magnet 72 in the second direction D2. Indeed, FIG. 18 shows the first armature 62 having been biased to its first position as shown in FIG. 9, and it shows the second armature 70 being biased toward, but not having been biased to, its second position. Following the second transition state of FIG. 18, the oscillator 10 returns to the first initial pressurization state of FIG. 11.

One skilled in the art would understand how to set the frequency of oscillation of the oscillator 10 as shown, for example, in FIGS. 11-18 as a function of various factors, for example, the fluid supply pressure, the sizes of the first and second accumulators A1, A2, and the movable surface area, the thickness, and the material of the first and second movable pressure barriers 42, 52, among others. The frequency of oscillation could be symmetric (the dwell time in position 1 may be the same as the dwell time in position 2) or asymmetric (the dwell time in position 1 may be different than the dwell time in position 2).

Also, one skilled in the art would recognize that the pressurization period and frequency for the first external accumulator A1 is a function of the rate of pressurization of the first pilot chamber 26B, and that the pressurization period and frequency for the second external accumulator A2 is a function of the rate of pressurization of the second pilot chamber 28B, among other factors. As such, one skilled in the art would recognize that the pressurization period and frequency of the first and second external accumulators A1, A2 may be selected as desired and that the pressurization period and frequency of the first external accumulator A1 may be the same as or different from the pressurization period and frequency of the second external accumulator A2. One skilled in the art would recognize that the oscillator 10 could be configured to charge either or both of the first and second external accumulators A1, A2 to pressures higher than the first and/or second pilot chamber 26B, 28B transition pressures by appropriate selection of, for example without limitation, the magnetic field strength(s) of the first and second magnets 64, 72 (and thus the biasing force between the first and second magnets 64, 72), the material and movable surface area of the first and/or second movable pressure barriers 42, 52, and the size of one or more internal fluid channels and and/or flow-restricting orifices within the oscillator 10.

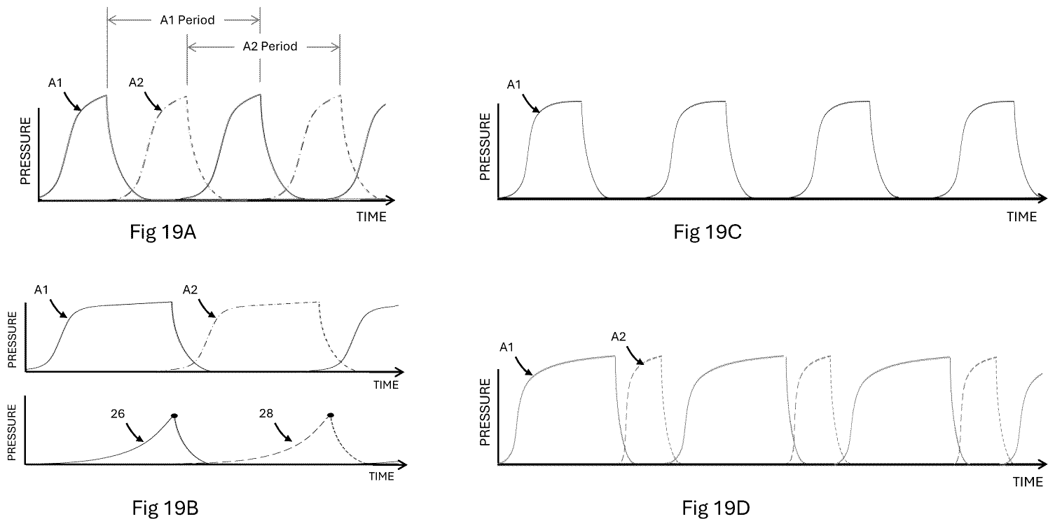

FIGS. 19A-19D are timing diagrams showing illustrative pressurization and venting of ones of the first and second external accumulators A1, A2 resulting from illustrative oscillations of the oscillator 10 as functions of time. More specifically, FIG. 19A shows timing of pressurization and venting of the first and second external accumulators A1, A2 as a function of time according to a first illustrative embodiment further to corresponding oscillation of the oscillator 10, wherein the pressurization period and frequency of the first external accumulator A1 is the same as the pressurization period and frequency of the second external accumulator A2. FIG. 19B shows timing of pressurization and venting of the first and second external accumulators A1, A2 as a function of time according to a second illustrative embodiment further to corresponding oscillation of the oscillator 10, wherein the pressurization period and frequency of the first external accumulator A1 is the same as the pressurization period and frequency of the second external accumulator A2. FIG. 19B also shows timing of corresponding pressurization and venting of the first and second pilot chambers 26B, 28B of the oscillator 10 according to the second illustrative embodiment. FIG. 19C shows timing of pressurization and venting of the first external accumulator A1 as a function of time according to a third illustrative embodiment further to corresponding oscillation of the oscillator 10. In the FIG. 19C embodiment, the second external accumulator A2 is omitted and the second bi-directional fluid port 18 is omitted or plugged. FIG. 19D shows timing of pressurization and venting of the first and second external accumulators A1, A2 as a function of time according to a fourth illustrative embodiment further to corresponding oscillation of the oscillator 10, wherein the pressurization period and frequency of the first external accumulator A1 is different from the pressurization period and frequency of the second external accumulator A2.

As mentioned above, either or both of the first and second bi-directional ports 16, 18 may be omitted or plugged. In such embodiments, the respective one or ones of the first and second external accumulators A1, A2 also would be omitted. In such embodiments, the operation of the flow oscillator 10 may similar to that described above, except that the flow control valve would not communicate fluid with the omitted one or ones of the first and second external accumulators A1, A2, and the structures of the omitted or plugged one or ones of the first and second bi-directional ports 16, 18 and the omitted one or ones of the first and second external accumulators A1, A2 (and associated fluid conduits) would not be factors contributing to the oscillation frequency of the oscillator 10. Accordingly, in such embodiments, the size of the pilot chambers corresponding to the omitted or plugged one(s) of the first and second bi-directional ports 16, 18, as discussed further above, and the size(s) of the flow restrictors corresponding to the omitted or plugged one(s) of the first and second bi-directional ports 16, 18 may be particularly relevant to (and may be the predominant factors in) achieving desired oscillation frequency characteristics for the oscillator 10, as would be understood by one skilled in the art.

As also mentioned above, either or both of the first and second bi-directional ports 16, 18 may deadheaded, for example without limitation, by plugging a respective one or ones of fluid conduits connected thereto, external to the housing 12. In such embodiments, the structures of the deadheaded one or ones of the first and second bi-directional ports 16, 18 and the fluid conduits connected thereto may remain factors contributing to the oscillation frequency of the oscillator 10. Nevertheless, in such embodiments, the size of the pilot chamber 26B, 28B corresponding to the omitted or plugged one(s) of the first and second bi-directional ports 16, 18 and the size(s) of the flow restrictors corresponding to the omitted or plugged one(s) of the first and second bi-directional ports 16, 18 may be particularly relevant to (and may be the predominant factors in) achieving desired oscillation frequency characteristics for the oscillator 10, as discussed further above, and as would be understood by one skilled in the art.

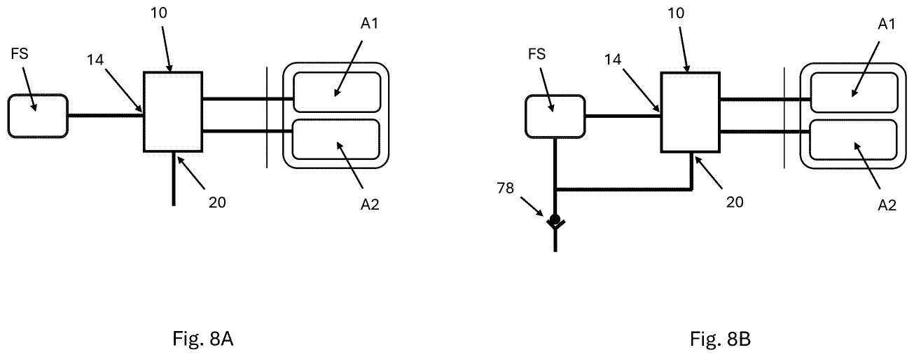

The foregoing description of operation of the oscillator 10 is directed to an embodiment wherein the exhaust port 20 is fluidly coupled to an environment surrounding the oscillator 10, and wherein the environment may be the atmosphere at ambient pressure. In embodiments, the exhaust port 20 may by fluidly coupled to an environment other than the atmosphere, wherein the environment may be at a pressure other than ambient pressure. For example, as shown in FIG. 8B, the exhaust port 20 may be coupled to an environment at a pressure greater than ambient pressure or less than ambient pressure, for example, a vacuum. In an embodiment, the source of pressurized fluid FS could be a fluid pump, for example a pneumatic pump, having a fluid inlet and a fluid outlet. The fluid inlet port 14 of the oscillator 10 could be fluidly coupled to the fluid outlet of the fluid pump, and the exhaust port 20 of the oscillator 10 could be fluidly coupled to the fluid inlet of the fluid pump. Thus, the fluid pump could draw a vacuum on the exhaust port 20 of the oscillator 10, while simultaneously providing pressurized fluid to the fluid inlet port 14 of the oscillator 10. Such an arrangement may facilitate evacuation of one or more external accumulators, for example, one or both of the first and second external accumulators A1, A2, connected to one or both of the bi-directional ports 16, 18 during operation of the oscillator 10. A vacuum breaker, for example, a calibrated check valve, could be provided in fluid communication with the pump inlet to provide make up fluid to the pump inlet. In any event, the pressure at the exhaust port 20 would be less than the pressure of the fluid provided to the fluid inlet port 14.

FIGS. 20-27 show illustrative embodiments of mechanical ventilators including the oscillator 10 and one or more respiratory interfaces connected to either or both of the first bi-directional port 16 and the second bi-directional port 18 in fluid communication therewith. Each such respiratory interface may be embodied in any suitable form configured to provide pressurized breathing to a user's lung or lungs or otherwise to the user's respiratory system. For example without limitation, the respiratory interface may be embodied as a positive pressure face mask (configured to provide pressurized breathing air to a user's mouth or nose or both) or a tracheal tube configured for intubation into a user, as will be discussed further below. Such a tracheal tube may be, for example without limitation, an endotracheal tube, a tracheostomy tube, or a nasotracheal tube.

FIG. 20 shows schematically a first illustrative embodiment of a mechanical ventilator 100 including the oscillator 10 and a first respiratory interface 102 fluidly connected to the first bi-directional port 16 of the oscillator 10. The second bi-directional port 18 is plugged or blocked or otherwise deadheaded or eliminated. The fluid exhaust port 20 is fluidly connected to an exhaust region E external to the oscillator 10. The exhaust region E may be an environment surrounding the mechanical ventilator 100. In embodiments, the exhaust region E may be a return air collection system or filtering apparatus (not shown) integral with or otherwise operably associated with the mechanical ventilator 100, as would be understood by one skilled in the art.

The fluid supply port 14 is fluidly connected to a source of pressurized breathing air BA (which may be modified with medicaments or otherwise as desired). The source of pressurized breathing air BA may be any suitable source of pressurized breathing air, for example without limitation, a hospital's pressurized air system or a self-contained, pressurized air canister. The source of pressurized breathing air BA may be configured to provide breathing air to the oscillator 10 and the mechanical ventilator 100 at a desired pressure. For example, the source of pressurized breathing air BA may be provided with a pressure regulator configured to provide breathing air to the mechanical ventilator 100 at a desired pressure. As shown in FIG. 21, embodiments of the mechanical ventilator 100 may further include an optional pressure regular PR configured to regulate the pressure of pressurized breathing air from the source of pressurized breathing air BA to the flow oscillator 10 to a desired pressure.

The oscillator 10 is configured (applying the principles discussed above) to oscillate in a manner that provides breathing air from the source of pressurized breathing air BA to a user according to a predetermined breathing cycle (or rhythm). Such a predetermined breathing cycle may include a predetermined inhalation period during which breathing air is provided from the source of pressurized breathing air BA to a user via the oscillator 10 and the first respiratory interface 102, and a predetermined exhalation period during which spent breathing air may be provided from the user to the exhaust region E via the first respiratory interface 102 and the oscillator 10. The predetermined inhalation period may be shorter than, longer than, or the same as the predetermined exhalation period. Non-limiting, illustrative breathing cycles are shown in FIGS. 25A-25C.

In operation, the first respiratory interface 102 may be applied to a user. For example, without limitation, a positive-pressure face mask may be fitted upon the user, or the user may be intubated with a tracheal tube, as suggested above. The oscillator 10 is charged with pressurized breathing air from the source of pressurized breathing air BA. When charged with pressurized breathing air, the oscillator 10 oscillates between a first internal alignment and a second internal alignment, according to the predetermined breathing cycle, further to the principles discussed above. In the first internal alignment, the oscillator 10 enables pressurized breathing air from the source of pressurized breathing air BA to be provided through the oscillator 10 (via the fluid supply port 14, the first armature chamber 22, the first fluid channel 30, and the first bi-directional port 16), to the first respiratory interface 102, and thereby to the user. In the second internal alignment, the oscillator 10 disables provision of pressurized breathing air thorough the oscillator 10 to the user, and it enables the user to exhale spent breathing air through the first respiratory interface 102 and the oscillator 10 (via the first bi-directional port 16, the first and third fluid channels 30, 34, the first pilot chamber 26, the second armature chamber 24, the fluid exhaust channel 20C and the fluid exhaust port 20) to the exhaust region E.

FIG. 22 shows schematically a mechanical ventilator 200 similar to the mechanical ventilator 100 of FIG. 20 but further including a second respiratory interface fluidly connected to the second bi-directional port 18 of the oscillator 10. In all other material respects, the mechanical ventilator 200 may be the same as the mechanical ventilator 100.

More specifically, the mechanical ventilator 200 includes a first respiratory interface 202 fluidly connected to the first bi-directional port 16 of the oscillator 10, and a second respiratory interface 204 fluidly connected to the second bi-directional port 18 of the oscillator 10. The second respiratory interface 204 may be embodied in the same form as the first respiratory interface 202 or in any other suitable form. For example, without limitation, the first respiratory interface 202 could be any of a positive pressure face mask, an endotracheal tube, a tracheostomy tube, or a nasotracheal tube, and the second respiratory interface 204 could be any of a positive pressure face mask, an endotracheal tube, a tracheostomy tube, or a nasotracheal tube.

In operation, the first respiratory interface 202 may be applied to a first user and the second respiratory 204 may be applied to a second user, as discussed in connection with the first respiratory interface 102 of the mechanical ventilator 100 of FIG. 20. The oscillator 10 is charged with pressurized breathing air from the source of pressurized breathing air BA. When charged with pressurized breathing air, the oscillator 10 oscillates between a first internal alignment and a second internal alignment according to the predetermined breathing cycle, further to the principles discussed above.

In the first internal alignment, the oscillator 10 enables pressurized breathing air from the source of pressurized breathing air BA to be provided through the oscillator 10 (via the fluid supply port 14, the first armature chamber 22, the first fluid channel 30, and the first bi-directional port 16), to the first respiratory interface 202, and thereby to the first user. Also in the first internal alignment, the oscillator 10 disables provision of pressurized breathing air thorough the oscillator 10 to the second user, and it enables the second user to exhale spent breathing air through the second respiratory interface 204 and the oscillator 10 (via the second bi-directional port 18, the second and fourth fluid channels 32, 36, the second pilot chamber 28, the second armature chamber 24, the fluid exhaust channel 20C and the fluid exhaust port 20) to the exhaust region E.

In the second internal alignment, the oscillator 10 disables provision of pressurized breathing air thorough the oscillator 10 to the first user, and it enables the first user to exhale spent breathing air through the first respiratory interface 202 and the oscillator 10 (via the first bi-directional port 16, the first and third fluid channels 30, 34, the first pilot chamber 26, the second armature chamber 24, the fluid exhaust channel 20C and the fluid exhaust port 20) to the exhaust region E. Also in the second internal alignment, the oscillator 10 enables pressurized breathing air from the source of pressurized breathing air BA to be provided through the oscillator 10 (via the fluid supply port 14, the first armature chamber 22, the second fluid channel 32, and the second bi-directional port 18), to the second respiratory interface 204, and thereby to the second user.

In the FIG. 22 embodiment, the predetermined breathing cycle for the second respiratory interface 204 is the substantial inverse of the predetermined breathing cycle for the first respiratory interface 202, as would be recognized by one skilled in the art. More specifically, the duration of the inhale portion of the predetermined breathing cycle for the first respiratory interface 202 is substantially the same as the duration of the exhale portion of the predetermined breathing cycle for the second respiratory interface 204, and the duration of the exhale portion of the predetermined breathing cycle for the first respiratory interface 202 is substantially the same as the duration of the inhale portion of the predetermined breathing cycle for the second respiratory interface 204.

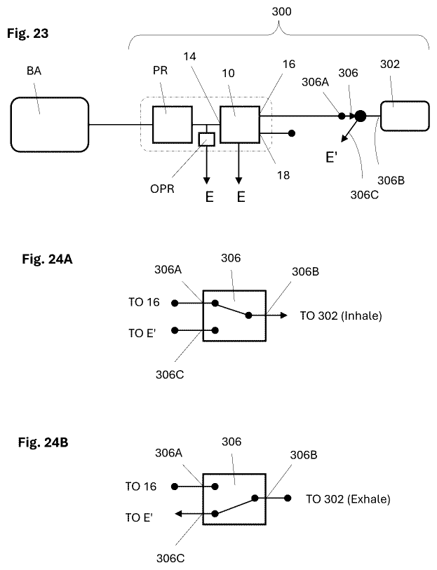

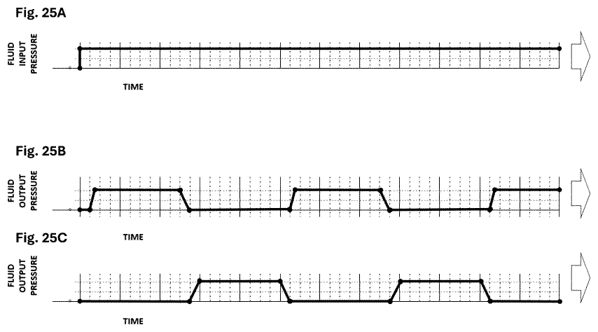

FIG. 23 shows schematically a mechanical ventilator 300 similar to the mechanical ventilator 100 but further including an optional pressure regulator PR fluidly connected between the source of breathing air BA and the fluid inlet port 14 of the flow oscillator 10, an optional overpressure relief device OPR fluidly connected between the pressure regulator PR and the flow oscillator 10, and a first directional control valve 600 fluidly connected between the first bi-directional port 16 of the oscillator 10 and the first respiratory interface. In all other material respects, the mechanical ventilator 300 may be the same as the mechanical ventilator 100.

More specifically, the mechanical ventilator 300 includes a first respiratory interface 302 and a first directional control valve 306. The first directional control valve 306 includes a fluid inlet port 306A, a bi-directional fluid port 306B, and a fluid outlet port 306C. The fluid inlet port 306A of the first directional control valve 306 is fluidly connected to the first bi-directional port of the oscillator 10. The bi-directional fluid port 306B of the first directional control valve 306 is fluidly connected to the first respiratory interface 302. The fluid outlet port 306C of the first directional control valve 306 is fluidly connected to an exhaust region E′ external to the first directional control valve 306. The exhaust region E′ may be an environment surrounding the mechanical ventilator 100. In embodiments, the exhaust region E′ may be a return air collection system or filtering apparatus (not shown) integral with or otherwise operably associated with the mechanical ventilator 100, as would be understood by one skilled in the art. The exhaust region E′ may be, but need not be, the same exhaust region as the exhaust region E.

In embodiments including the optional pressure regulator PR and the optional overpressure relief device OPR, the overpressure relief device OPR typically would be installed downstream of the optional pressure regulator PR (that is, between the optional pressure regulator PR and the oscillator 10). The optional overpressure relief device OPR is shown as fluidly connected between the pressure regulator PR and the oscillator 10. In embodiments, the overpressure relief device OPR (or one or more additional overpressure relief devices OPR) may be provided elsewhere between the source of pressurized breathing air BA and the first respiratory interface 102 in fluid connection therewith to protect a user from an overpressure condition that might occur in the event of a failure to regulate the pressure of pressurized breathing air BA supplied to the mechanical ventilator 10 from the source of pressurized breathing air BA to a safe pressure. Such overpressure relief devices OPR typically would be configured to change state from a normal state to an overpressure-relieving state at a nominally higher pressure than the desired, regulated pressure. In the overpressure-relieving state, the overpressure relief devices OPR typically would be configured to relieve overpressurized breathing air to the exhaust region E. Such overpressure relief devices OPR may be embodied in any suitable form, for example without limitation, as pressure relief valves, calibrated check valves, or rupture discs.

FIGS. 24A and 24B show schematically the first directional control valve 306 in first and second operational states, respectively. In the first operational state shown in FIG. 24A, the first directional control valve 306 is configured to enable flow therethrough from the fluid inlet port 306A thereof to the bi-directional port 306B thereof, and to thereby enable flow from the first bi-directional port 16 of the oscillator 10 to the first respiratory interface 302. Also in the first operational state, the first directional control valve 306 is configured to disable flow therethrough from the fluid inlet port 306A thereof and the bi-directional port 306B thereof to the fluid outlet port 306C thereof, and to thereby disable flow from of the first bi-directional port 16 of the oscillator 10 and the first respiratory interface 302 to the exhaust region E′ external to the first directional control valve 306. This first operational state corresponds to an inhale portion of the predetermined breathing cycle.

In the second operational state shown in FIG. 24B, the first directional flow control valve 306 is configured to enable flow therethrough from the bi-directional port 306B thereof to the fluid outlet port 306C thereof, and to thereby enable flow from the first respiratory interface 302 to the exhaust region E′ external to the first directional control valve 306. Also in the second operational state, the first directional flow control valve 306 is configured to disable flow therethrough from the bi-directional port 306B thereof and the fluid outlet port 306C thereof to the inlet port 306A thereof, and to thereby disable flow from the first respiratory interface 302 and the exhaust region E′ to the first bi-directional port 16 of the oscillator 10. This second operational state corresponds to an exhale portion of the predetermined breathing cycle.

The first directional control 306 is configured to adopt one of the first operational state and the second operational state in response to fluid pressure at the fluid inlet port 306A thereof. More specifically, when the fluid pressure at the fluid inlet port 306A of the first directional control valve 306 is relatively high, for example, when the oscillator 10 is aligned to provide pressurized breathing air through the first bi-directional port 16 to the first directional control valve 306, the first directional control valve 306 adopts the first operational state. Conversely, when the fluid pressure at the fluid inlet port 306A of the first directional control valve 306 is relatively low, for example, when the oscillator 10 is aligned to vent pressurized breathing air from between the first bi-directional port 16 and the first directional control valve 306 through the oscillator 10 to the exhaust region E, the first directional control valve 306 adopts the second operational state. As such, the first directional control valve 306 generally is in the first operational state when the oscillator 10 is in a first internal alignment, as discussed further below, and the first directional control valve 306 generally is in a second operational state when the oscillator 10 is in the second internal alignment, as discussed further below.

In operation, the first respiratory interface 302 may be applied to a first user, as discussed in connection with the first respiratory interface 102 of the mechanical ventilator 100 of FIG. 20. The oscillator 10 is charged with pressurized breathing air from the source of pressurized breathing air BA. When charged with pressurized breathing air, the oscillator 10 oscillates between the first internal alignment and the second internal alignment according to the predetermined breathing cycle, further to the principles discussed above.

In the first internal alignment, the oscillator 10 enables pressurized breathing air from the source of pressurized breathing air BA to be provided through the oscillator 10 (via the fluid supply port 14, the first armature chamber 22, the first fluid channel 30, and the first bi-directional port 16) to the fluid inlet port 306A of the first directional control valve 306. The pressurized breathing air imparts relatively high pressure at the fluid inlet port 306A of the first directional control valve 306, thus causing the first directional control valve 306 to adopt the first operational state, further to the principles discussed above. In the first operational state, the first directional control valve 306 enables the pressurized breathing air to be provided therethrough (via the fluid inlet port 306A thereof and the bi-directional port 306B thereof) to the first respiratory interface 302, and thereby to the user. Also in the first operational state, the first directional control valve 306 disables flow of the pressurized breathing air through the fluid outlet port 306C thereof.

In the second internal alignment, the oscillator 10 disables provision of pressurized breathing air thorough the oscillator 10 to the first directional control valve 306, and it enables venting of pressurized breathing air at the fluid inlet port 306A of the first directional control valve 306 through the oscillator 10, as discussed above. Such venting of pressurized breathing air at the fluid inlet port 306A lowers the pressure there to a relatively low pressure, thus causing the first directional control valve 306 to adopt the second operational state, further to the principles discussed above. In the second operational state, the first directional control valve 306 enables the user to exhale spent breathing air through the first respiratory interface 302 and the first directional control valve 306 (via the bi-directional port 306B thereof and the fluid outlet port 306C thereof) to the exhaust region E′. Also in the second operational state, the first directional control valve 306 disables flow of the spent breathing air through the fluid inlet port 306A thereof.

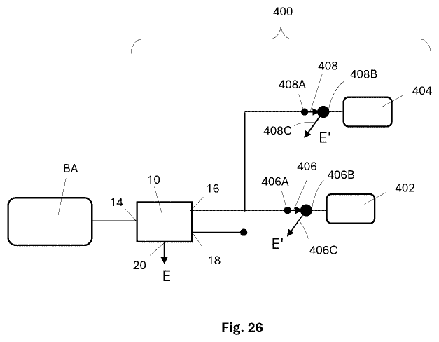

FIG. 26 shows schematically a mechanical ventilator 400 similar to the mechanical ventilator 300 but further including a second respiratory interface and a second directional control valve fluidly connected to the first bi-directional port 16 in parallel with a first respiratory interface and a first directional control valve. In all other material respects, the mechanical ventilator 400 may be the same as the mechanical ventilator 300.

More specifically, the mechanical ventilator 400 includes a first respiratory interface 402 and a first directional control valve 406 analogous to the first directional control valve 306 of the mechanical ventilator 300. The first directional control valve 406 thus includes a fluid inlet port 406A, a bi-directional fluid port 406B, and a fluid outlet port 406C. The fluid inlet port 406A of the first directional control valve 306 is fluidly connected to the first bi-directional port of the oscillator 10. The bi-directional fluid port 406B of the first directional control valve 406 is fluidly connected to the first respiratory interface 402. The fluid outlet port 402C of the first directional control valve 406 is fluidly connected to an exhaust region E′ external to the first directional control valve 406. The exhaust region E′ may be an environment surrounding the mechanical ventilator 100. In embodiments, the exhaust region E′ may be a return air collection system or filtering apparatus (not shown) integral with or otherwise operably associated with the mechanical ventilator 100, as would be understood by one skilled in the art. The exhaust region E′ may be, but need not be, the same exhaust region as exhaust region E.

The mechanical ventilator 400 also includes a second respiratory interface 404 and a second directional control valve 408. The second respiratory interface 404 may be embodied in the same form as the first respiratory interface 402 or in any other suitable form. For example, without limitation, the first respiratory interface 402 could be any of a positive pressure face mask, an endotracheal tube, a tracheostomy tube, or a nasotracheal tube, and the second respiratory interface 404 could be any of a positive pressure face mask, an endotracheal tube, a tracheostomy tube, or a nasotracheal tube.

The second directional control valve 408 is analogous to the first directional control valve 306 of the mechanical ventilator 300. The second directional control valve 408 thus includes a fluid inlet port 408A, a bi-directional fluid port 408B, and a fluid outlet port 408C. The fluid inlet port 408A of the second directional control valve 408 is fluidly connected to the first bi-directional port of the oscillator 10 in parallel with the first respiratory interface 402 and the first directional control valve 406. The bi-directional fluid port 408B of the second directional control valve 408 is fluidly connected to the second respiratory interface 406. The fluid outlet port 408C of the second directional control valve 408 is fluidly connected to the exhaust region E′ or to another exhaust region external to the second directional control valve 408.

The manner of operation of the mechanical ventilator 400 is substantially similar to the operation of the mechanical ventilator 300. In operation, the first respiratory interface 402 may be applied to a first user, and the second respiratory interface 404 may be applied to a second user, as discussed in connection with the first respiratory interface 102 of the mechanical ventilator 100 of FIG. 20. The oscillator 10 is charged with pressurized breathing air from the source of pressurized breathing air BA. When charged with pressurized breathing air, the oscillator 10 oscillates between the first internal alignment and the second internal alignment according to the predetermined breathing cycle, further to the principles discussed above.

In the first internal alignment, the oscillator 10 enables pressurized breathing air from the source of pressurized breathing air BA to be provided through the oscillator 10 (via the fluid supply port 14, the first armature chamber 22, the first fluid channel 30, and the first bi-directional port 16) to the fluid inlet port 406A of the first directional control valve 406. The pressurized breathing air imparts relatively high pressure at the fluid inlet port 406A of the first directional control valve 406, thus causing the first directional control valve 406 to adopt the first operational state, further to the principles discussed above. In the first operational state, the first directional control valve 406 enables the pressurized breathing air to be provided therethrough (via the fluid inlet port 406A thereof and the bi-directional port 406B thereof) to the first respiratory interface 402, and thereby to the first user. Also in the first operational state, the first directional control valve 406 disables flow of the pressurized breathing air through the fluid outlet port 406C thereof.

At the same time, the oscillator 10 enables pressurized breathing air from the source of pressurized breathing air BA to be provided through the oscillator 10 (via the fluid supply port 14, the first armature chamber 22, the first fluid channel 30, and the first bi-directional port 16) to the fluid inlet port 408A of the second directional control valve 408. The pressurized breathing air imparts relatively high pressure at the fluid inlet port 408A of the second directional control valve 408, thus causing the second directional control valve 408 to adopt the first operational state, further to the principles discussed above. In the first operational state, the second directional control valve 408 enables the pressurized breathing air to be provided therethrough (via the fluid inlet port 408A thereof and the bi-directional port 408B thereof) to the second respiratory interface 404, and thereby to the second user. Also in the first operational state, the second directional control valve 406 disables flow of the pressurized breathing air through the fluid outlet port 406C thereof.

In the second internal alignment, the oscillator 10 disables provision of pressurized breathing air thorough the oscillator 10 to the first directional control valve 406, and it enables venting of pressurized breathing air at the fluid inlet port 406A of the first directional control valve 406 through the oscillator 10, as discussed above. Such venting of pressurized breathing air at the fluid inlet port 406A lowers the pressure there to a relatively low pressure, thus causing the first directional control valve 406 to adopt the second operational state, further to the principles discussed above. In the second operational state, the first directional control valve 406 enables the user to exhale spent breathing air through the first respiratory interface 402 and the first directional control valve 406 (via the bi-directional port 406B thereof and the fluid outlet port 406C thereof) to the exhaust region E′. Also in the second operational state, the first directional control valve 406 disables flow of the spent breathing air through the fluid inlet port 406A thereof.

At the same time, the oscillator 10 disables provision of pressurized breathing air thorough the oscillator 10 to the second directional control valve 408, and it enables venting of pressurized breathing air at the fluid inlet port 408A of the second directional control valve 408 through the oscillator 10, as discussed above. Such venting of pressurized breathing air at the fluid inlet port 408A lowers the pressure there to a relatively low pressure, thus causing the second directional control valve 408 to adopt the second operational state, further to the principles discussed above. In the second operational state, the second directional control valve 408 enables the user to exhale spent breathing air through the second respiratory interface 404 and the second directional control valve 408 (via the bi-directional port 408B thereof and the fluid outlet port 408C thereof) to the exhaust region E′. Also in the second operational state, the second directional control valve 408 disables flow of the spent breathing air through the fluid inlet port 408A thereof.

In embodiments, either or both of the first and second directional control valves 406, 408 could be omitted, and the corresponding ones of the first and second respiratory interfaces 402, 404 could be fluidly connected to the first bi-directional port 16 of the oscillator 10, for example, in a manner similar to that in which the first respiratory interface 102 is fluidly connected to the control valve of the mechanical ventilator 100. In such embodiments, the operation of the mechanical ventilator 400 would be substantially similar to that of the mechanical ventilator 100 as applied to the one(s) of the first and second respiratory interfaces 402, 404 lacking a corresponding directional control valve.

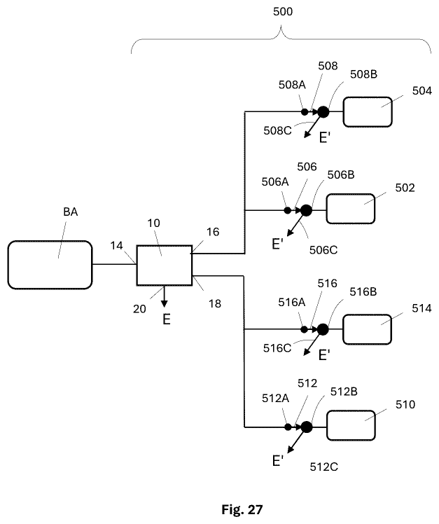

FIG. 27 shows schematically a mechanical ventilator 500 similar to the mechanical ventilator 400 but further including third and fourth respiratory interfaces and corresponding third and fourth directional control valves fluidly connected to the second bi-directional port 18 of the oscillator 10. In all other material respects, the mechanical ventilator 500 may be the same as the mechanical ventilator 400.

More specifically, the mechanical ventilator 500 includes a first respiratory interface 502 and a first directional control valve 506 analogous to the first directional control valve 306 of the mechanical ventilator 300. The first directional control valve 506 thus includes a fluid inlet port 506A, a bi-directional fluid port 506B, and a fluid outlet port 506C. The fluid inlet port 506A of the first directional control valve 506 is fluidly connected to the first bi-directional port 16 of the oscillator 10. The bi-directional fluid port 506B of the first directional control valve 506 is fluidly connected to the first respiratory interface 502. The fluid outlet port 502C of the first directional control valve 506 is fluidly connected to an exhaust region E′ external to the first directional control valve 506. The exhaust region E′ may be an environment surrounding the mechanical ventilator 100. In embodiments, the exhaust region E′ may be a return air collection system or filtering apparatus (not shown) integral with or otherwise operably associated with the mechanical ventilator 100, as would be understood by one skilled in the art. The exhaust region E′ may be, but need not be, the same exhaust region as exhaust region E.

The mechanical ventilator 500 also includes a second respiratory interface 504 and a second directional control valve 508. The second respiratory interface 504 may be embodied in the same form as the first respiratory interface 502 or in any other suitable form. For example, without limitation, the first respiratory interface 502 could be any of a positive pressure face mask, an endotracheal tube, a tracheostomy tube, or a nasotracheal tube, and the second respiratory interface 504 could be any of a positive pressure face mask, an endotracheal tube, a tracheostomy tube, or a nasotracheal tube. The second directional control valve 508 is analogous to the first directional control valve 306 of the mechanical ventilator 300. The second directional control valve 508 thus includes a fluid inlet port 508A, a bi-directional fluid port 508B, and a fluid outlet port 508C. The fluid inlet port 508A of the second directional control valve 508 is fluidly connected to the first bi-directional port 16 of the oscillator 10 in parallel with the first respiratory interface 502 and the first directional control valve 406. The bi-directional fluid port 508B of the second directional control valve 508 is fluidly connected to the second respiratory interface 506. The fluid outlet port 508C of the second directional control valve 508 is fluidly connected to the exhaust region E′ or to another exhaust region external to the second directional control valve 508.

The mechanical ventilator 500 further includes a third respiratory interface 510 and a third directional control valve 512. The third respiratory interface 510 may be embodied in the same form as the first respiratory interface 502 or in any other suitable form. For example, without limitation, the first respiratory interface 502 could be any of a positive pressure face mask, an endotracheal tube, a tracheostomy tube, or a nasotracheal tube, and the second respiratory interface 504 could be any of a positive pressure face mask, an endotracheal tube, a tracheostomy tube, or a nasotracheal tube.

The third directional control valve 512 is analogous to the first directional control valve 306 of the mechanical ventilator 300. The third directional control valve 512 thus includes a fluid inlet port 512A, a bi-directional fluid port 512B, and a fluid outlet port 512C. The fluid inlet port 512A of the third directional control valve 512 is fluidly connected to the second bi-directional port 18 of the oscillator 10. The bi-directional fluid port 512B of the third directional control valve 506 is fluidly connected to the third respiratory interface 510. The fluid outlet port 512C of the third directional control valve 512 is fluidly connected to an exhaust region E′ external to the third directional control valve 512. The exhaust region E′ may be an environment surrounding the mechanical ventilator 100. In embodiments, the exhaust region E′ may be a return air collection system or filtering apparatus (not shown) integral with or otherwise operably associated with the mechanical ventilator 100, as would be understood by one skilled in the art. The exhaust region E′ may be, but need not be, the same exhaust region as exhaust region E.

The mechanical ventilator 500 also includes a fourth respiratory interface 514 and a fourth directional control valve 516. The fourth respiratory interface 514 may be embodied in the same form as the first respiratory interface 502 or in any other suitable form. For example, without limitation, the first respiratory interface 502 could be any of a positive pressure face mask, an endotracheal tube, a tracheostomy tube, or a nasotracheal tube, and the fourth respiratory interface 514 could be any of a positive pressure face mask, an endotracheal tube, a tracheostomy tube, or a nasotracheal tube.

The fourth directional control valve 516 is analogous to the first directional control valve 306 of the mechanical ventilator 300. The fourth directional control valve 516 thus includes a fluid inlet port 516A, a bi-directional fluid port 516B, and a fluid outlet port 516C. The fluid inlet port 516A of the fourth directional control valve 516 is fluidly connected to the second bi-directional port 18 of the oscillator 10 in parallel with the third respiratory interface 510 and the third directional control valve 512. The bi-directional fluid port 516B of the fourth directional control valve 516 is fluidly connected to the fourth respiratory interface 514. The fluid outlet port 516C of the fourth directional control valve 516 is fluidly connected to the exhaust region E′ or to another exhaust region external to the fourth directional control valve 516.

In operation, the first respiratory interface 502 may be applied to a first user, the second respiratory interface 504 may be applied to a second user, the third respiratory interface 510 may be applied to a third user, and the fourth respiratory interface 514 may be applied to a second user as discussed in connection with the first respiratory interface 102 of the mechanical ventilator 100 of FIG. 20. The oscillator 10 is charged with pressurized breathing air from the source of pressurized breathing air BA. When charged with pressurized breathing air, the oscillator 10 oscillates between the first internal alignment and the second internal alignment according to the predetermined breathing cycle, further to the principles discussed above.

In the first internal alignment, the oscillator 10 enables pressurized breathing air from the source of pressurized breathing air BA to be provided through the oscillator 10 (via the fluid supply port 14, the first armature chamber 22, the first fluid channel 30, and the first bi-directional port 16) to the fluid inlet port 506A of the first directional control valve 506. The pressurized breathing air imparts relatively high pressure at the fluid inlet port 506A of the first directional control valve 506, thus causing the first directional control valve 506 to adopt the first operational state, further to the principles discussed above. Accordingly, the first directional control valve 506 enables the pressurized breathing air to be provided therethrough (via the fluid inlet port 506A thereof and the bi-directional port 506B thereof) to the first respiratory interface 502, and thereby to the first user. Also, the first directional control valve 506 disables flow of the pressurized breathing air through the fluid outlet port 506C thereof.

At the same time, the oscillator 10 enables pressurized breathing air from the source of pressurized breathing air BA to be provided through the oscillator 10 (via the fluid supply port 14, the first armature chamber 22, the first fluid channel 30, and the first bi-directional port 16) to the fluid inlet port 508A of the second directional control valve 508. The pressurized breathing air imparts relatively high pressure at the fluid inlet port 508A of the second directional control valve 508, thus causing the second directional control valve 508 to adopt the first operational state, further to the principles discussed above. Accordingly, the second directional control valve 508 enables the pressurized breathing air to be provided therethrough (via the fluid inlet port 508A thereof and the bi-directional port 508B thereof) to the second respiratory interface 504, and thereby to the second user. Also, the second directional control valve 506 disables flow of the pressurized breathing air through the fluid outlet port 506C thereof.

Also in the first alignment, the oscillator 10 disables provision of pressurized breathing air thorough the oscillator 10 to the third directional control valve 512, and it enables venting of pressurized breathing air at the fluid inlet port 512A of the third directional control valve 512 through the oscillator 10, as discussed above. Such venting of pressurized breathing air at the fluid inlet port 512A lowers the pressure there to a relatively low pressure, thus causing the third directional control valve 512 to adopt the second operational state, further to the principles discussed above. Accordingly, the third directional control valve 512 enables the third user to exhale spent breathing air through the third respiratory interface 510 and the third directional control valve 512 (via the bi-directional port 512B thereof and the fluid outlet port 512C thereof) to the exhaust region E′. Also, the third directional control valve 512 disables flow of the spent breathing air through the fluid inlet port 512A thereof.

At the same time, the oscillator 10 disables provision of pressurized breathing air thorough the oscillator 10 to the fourth directional control valve 516, and it enables venting of pressurized breathing air at the fluid inlet port 516A of the fourth directional control valve 516 through the oscillator 10, as discussed above. Such venting of pressurized breathing air at the fluid inlet port 516A lowers the pressure there to a relatively low pressure, thus causing the fourth directional control valve 516 to adopt the second operational state, further to the principles discussed above. Accordingly, the fourth control valve 516 enables the user to exhale spent breathing air through the fourth respiratory interface 514 and the fourth directional control valve 516 (via the bi-directional port 516B thereof and the fluid outlet port 516C thereof) to the exhaust region E′. Also, the fourth directional control valve 516 disables flow of the spent breathing air through the fluid inlet port 516A thereof.

In the second internal alignment, the oscillator 10 disables provision of pressurized breathing air thorough the oscillator 10 to the first directional control valve 506, and it enables venting of pressurized breathing air at the fluid inlet port 506A of the first directional control valve 506 through the oscillator 10, as discussed above. Such venting of pressurized breathing air at the fluid inlet port 506A lowers the pressure there to a relatively low pressure, thus causing the first directional control valve 506 to adopt the second operational state, further to the principles discussed above. Accordingly, the first directional control valve 506 enables the first user to exhale spent breathing air through the first respiratory interface 502 and the first directional control valve 506 (via the bi-directional port 506B thereof and the fluid outlet port 506C thereof) to the exhaust region E′. Also, the first directional control valve 506 disables flow of the spent breathing air through the fluid inlet port 506A thereof.

At the same time, the oscillator 10 disables provision of pressurized breathing air thorough the oscillator 10 to the second directional control valve 508, and it enables venting of pressurized breathing air at the fluid inlet port 508A of the second directional control valve 508 through the oscillator 10, as discussed above. Such venting of pressurized breathing air at the fluid inlet port 508A lowers the pressure there to a relatively low pressure, thus causing the second directional control valve 508 to adopt the second operational state, further to the principles discussed above. Accordingly, the second directional control valve 508 enables the user to exhale spent breathing air through the second respiratory interface 504 and the second directional control valve 508 (via the bi-directional port 508B thereof and the fluid outlet port 508C thereof) to the exhaust region E′. Also, the second directional control valve 508 disables flow of the spent breathing air through the fluid inlet port 508A thereof.

Also in the second internal alignment, the oscillator 10 enables pressurized breathing air from the source of pressurized breathing air BA to be provided through the oscillator 10 (via the fluid supply port 14, the first armature chamber 22, the first fluid channel 30, and the first bi-directional port 16) to the fluid inlet port 512A of the third directional control valve 512. The pressurized breathing air imparts relatively high pressure at the fluid inlet port 512A of the third directional control valve 512, thus causing the third directional control valve 512 to adopt the first operational state, further to the principles discussed above. Accordingly, the third directional control valve 512 enables the pressurized breathing air to be provided therethrough (via the fluid inlet port 512A thereof and the bi-directional port 512B thereof) to the third respiratory interface 510, and thereby to the third. Also, the third directional control valve 512 disables flow of the pressurized breathing air through the fluid outlet port 512C thereof.

At the same time, the oscillator 10 enables pressurized breathing air from the source of pressurized breathing air BA to be provided through the oscillator 10 (via the fluid supply port 14, the first armature chamber 22, the first fluid channel 30, and the first bi-directional port 16) to the fluid inlet port 516A of the fourth directional control valve 516. The pressurized breathing air imparts relatively high pressure at the fluid inlet port 516A of the fourth directional control valve 516, thus causing the fourth directional control valve 516 to adopt the first operational state, further to the principles discussed above. Accordingly, the fourth directional control valve 516 enables the pressurized breathing air to be provided therethrough (via the fluid inlet port 516A thereof and the bi-directional port 516B thereof) to the fourth respiratory interface 514, and thereby to the fourth user. Also, the fourth directional control valve 516 disables flow of the pressurized breathing air through the fluid outlet port 516C thereof.

In embodiments including plural respiratory interfaces, individual ones of the plural respiratory interfaces may be operable independent of the use of other ones of the plural respiratory interfaces. For example without limitation, any one or more of the first through fourth respiratory interfaces 502, 504, 510, 514 of the mechanical ventilator 500 may be operable in connection with one or more corresponding users, even if other ones of the first through fourth respiratory interfaces 502, 504, 510, 514 are not being used by corresponding users.

Embodiments wherein a single flow oscillator 10 serves plural of respiratory interfaces without corresponding, intervening, directional control valves, for example without limitation the FIG. 22 embodiment, may involve a bio-hazard risk due to potential cross-contamination of one or more of the plural respiratory interfaces by spent air routed through the flow control valve by one or more others of the plural respiratory interfaces. In such embodiments, bio-filters may be provided between the flow oscillator 10 and corresponding ones of the plural respiratory interfaces.

The disclosure illustrates and describes an oscillating flow controller internally configured to receive pressurized breathing air through a fluid input port and alternatingly provide pressurized breathing air output through first and second bi-directional ports. The oscillating flow controller is passive, that is, it requires no external power other than the pressurized breathing air to effect the oscillating output thereof. The benefits of the disclosure may be realized using flow controllers or flow control valves having other internal configurations that enable an oscillating output similar to that described herein.

The embodiments shown and described herein are illustrative and not limiting. One skilled in the art would recognize that features of any embodiment may be freely interchanged with features of other embodiments without departure from the scope of the appended claims. One skilled in the art also would recognize that any embodiment may be readily modified without departure from the scope of the appended claims.

1. A mechanical ventilator comprising:
an oscillating flow controller comprising a supply port, a first bi-directional port, and an exhaust port, wherein the oscillating flow controller is configured to receive pressurized breathing air through the supply port, selectively output the pressurized breathing air through the first bi-directional port, selectively receive the pressurized breathing air through the first-bi-directional port, and selectively output the pressurized breathing air though the exhaust port; and
a first respiratory interface having a first breathing air conduit, a first end of the first breathing air conduit fluidly coupled to the first bi-directional port and a second end of the first breathing air conduit configured to supply the breathing air from the first bi-directional port to a respiratory system of a first user,
wherein the oscillating flow controller further is configured to alternate between a first state in which the oscillating flow controller enables flow of the pressurized breathing air from the supply port through the first bi-directional port to the first breathing air conduit and disables flow of the pressurized breathing air from the supply port and the first bi-directional port to the exhaust port, and a second state in which the oscillating flow controller enables flow of the pressurized breathing air from the first breathing air conduit through the first bi-directional port to the exhaust port and disables flow of the pressurized breathing air from the supply port to the first bi-directional port and the exhaust port, and
wherein the oscillating flow controller further is configured to cyclically change state between the first state and the second state in response to the pressurized breathing air flowing therethrough.

2. The mechanical ventilator of claim 1, wherein the first respiratory interface is a first tracheal tube having a first end proximate the first bi-directional port and a second end configured for intubation into the first user.

3. The mechanical ventilator of claim 2, wherein the first tracheal tube is configured to cyclically transfer the pressurized breathing air from the first end to the second end and to periodically transfer spent breathing air from the second end to the first end.

4. The mechanical ventilator of claim 2, further comprising a first directional control valve fluidly connected between the first bi-directional port and the first tracheal tube,
wherein the first directional control valve comprises a first port fluidly connected to the first bi-directional port, a second port fluidly connected to the first tracheal tube, and a third port fluidly connected to an environment external to the mechanical ventilator, and
wherein the first directional control valve is operable between a first state in which the first directional control valve is configured to enable fluid flow from the first bi-directional port through the first port and the second port to the first tracheal tube and to disable fluid flow through the third port, and a second state in which the first directional control valve is configured to disable fluid flow through the first port and to enable fluid flow from the tracheal tube through the second port and the third port to the environment external to the mechanical ventilator.

5. The mechanical ventilator of claim 4, wherein the first directional control valve is controlled by fluid pressure between the first bi-directional port and the first port.

6. The mechanical ventilator of claim 5, wherein the first directional control valve is in the first state when the fluid pressure between the first bi-directional port and the first port is relatively high.

7. The mechanical ventilator of claim 6, wherein the first directional control valve is in the second state when the fluid pressure between the first bi-directional port and the first port is relatively low.

8. The mechanical ventilator of claim 1, further comprising a second respiratory interface fluidly connected to the first bi-directional port, wherein the second respiratory interface is configured to provide breathing air from the fluid supply port to a respiratory system of a second user.

9. The mechanical ventilator of claim 1, further comprising a second respiratory interface having a second breathing air conduit,
wherein the oscillating flow controller further comprises a second bi-directional port,
wherein the oscillating flow controller further is configured to selectively output the pressurized breathing air through the second bi-directional port and selectively receive the pressurized breathing air through the second bi-directional port,
wherein a first end of the second breathing air conduit is fluidly coupled to the second bi-directional port and a second end of the second breathing air conduit is configured to supply the breathing air from the second bi-directional port to a respiratory system of a second user, and
wherein the oscillating flow controller further is configured to: in the first state, disable flow of the pressurized breathing air from the supply port through the second bi-directional port to the second breathing air conduit and enable flow of the pressurized breathing air from the second breathing air conduit through the second bi-directional port to the exhaust port; and, in the second state enable flow of the pressurized breathing air through the second bi-directional port to the second breathing air conduit and disable flow of the pressurized breathing air from the second bi-directional port to the exhaust port.

10. The mechanical ventilator of claim 1, wherein the first respiratory interface is a positive-pressure face mask.

11. The mechanical ventilator of claim 1, wherein the oscillating flow controller is configured to change state between the first state and the second state according to a predetermined cycle corresponding to a predetermined breathing pattern.

12. A method of mechanical ventilation, comprising:
providing an oscillating flow controller comprising a supply port, a first bi-directional port, and an exhaust port, wherein the oscillating flow controller is configured to receive breathing air through the supply port, selectively output the breathing air through the first bi-directional port, selectively receive the breathing air through the first bi-directional port, and selectively output the breathing air through the exhaust port,
wherein the oscillating flow controller further is configured to alternate between a first state in which the oscillating flow controller enables flow of the breathing air from the supply port through the first bi-directional port to the breathing air conduit and disables flow of the breathing air from the supply port and the first bi-directional port to the exhaust port, and a second state in which the oscillating flow controller enables flow of the breathing air from the breathing air conduit through the first bi-directional port to the exhaust port and disables flow of the breathing air from the supply port to the first bi-directional port and the exhaust port, and
wherein the oscillating flow controller further is configured to cyclically change state between the first state and the second state in response to the breathing air flowing therethrough,
providing a first respiratory interface having a first breathing air conduit;
fluidly coupling a first end of the first breathing air conduit fluidly to the first bi-directional port;
fluidly coupling a second end of the first breathing air conduit to a respiratory system of a user; and
fluidly coupling the supply port to a source of pressurized breathing air.

13. The method of claim 12 wherein the first respiratory interface is a tracheal tube.

14. The method of claim 12 wherein the first respiratory interface is a positive pressure mask.

15. The method of claim 12, further comprising:
providing a second respiratory interface having a second breathing air conduit,
wherein the oscillating flow controller further comprises a second bi-directional port,
wherein the oscillating flow controller further is configured to selectively output the pressurized breathing air through the second bi-directional port and selectively receive the pressurized breathing air through the second bi-directional port,
wherein a first end of the second breathing air conduit is fluidly coupled to the second bi-directional port and a second end of the second breathing air conduit is configured to supply the breathing air from the second bi-directional port to a respiratory system of a second user, and
wherein the oscillating flow controller further is configured to: in the first state, disable flow of the pressurized breathing air from the supply port through the second bi-directional port to the second breathing air conduit and enable flow of the pressurized breathing air from the second breathing air conduit through the second bi-directional port to the exhaust port; and, in the second state enable flow of the pressurized breathing air through the second bi-directional port to the second breathing air conduit and disable flow of the pressurized breathing air from the second bi-directional port to the exhaust port.

16. The method of claim 12, wherein the oscillating flow controller is configured to change state between the first state and the second state according to a predetermined cycle corresponding to a predetermined breathing pattern.
